(19) United States
(12) Patent Application Publication
(10) Pub. No.: US 2026/0248866 A1
(43) Pub. Date: Aug. 27, 2026

(54) BACTERIOPHAGES, LYTIC AGENTS FOR SALMONELLA SPECIES, COMPOSITIONS, AND METHOD FOR CONTROLLING SALMONELLA SPECIES

(71) Applicants: KANEKA CORPORATION, Osaka (JP); RAKUNO GAKUEN UNIVERSITY, Hokkaido (JP); MITSUI & CO., LTD., Tokyo (JP)

(72) Inventors: Shinichi Yoshida, Shizuoka (JP); Nobuhiko Dojun, Shizuoka (JP); Hidetomo Iwano, Hokkaido (JP); Ryo Murata, Hokkaido (JP); Ikuo Uchida, Hokkaido (JP); Yoshifumi Hatanaka, Tokyo (JP)

(73) Assignees: KANEKA CORPORATION, Osaka (JP); RAKUNO GAKUEN UNIVERSITY, Hokkaido (JP); MITSUI & CO., LTD., Tokyo (JP)

(21) Appl. No.: 19/346,306

(22) Filed: Sep. 30, 2025

Related U.S. Application Data
(63) Continuation of application No. PCT/JP2024/013050, filed on Mar. 29, 2024.

(30) Foreign Application Priority Data
Mar. 31, 2023 (JP) 2023-057024
Mar. 31, 2023 (JP) 2023-057035
Mar. 31, 2023 (JP) 2023-057261
Mar. 31, 2023 (JP) 2023-057268
Mar. 31, 2023 (JP) 2023-057554
Mar. 31, 2023 (JP) 2023-057565
Mar. 31, 2023 (JP) 2023-057568
Mar. 31, 2023 (JP) 2023-057617

(51) Int. Cl. A61K 35/76; A61P 31/04; C07K 14/005

(52) U.S. Cl. CPC A61K 35/76; A61P 31/04; C07K 14/005; C12N 2795/00022; C12N 2795/00032

(57) ABSTRACT

An object of the present disclosure is to provide (i) a novel bacteriophage having lytic activity against a Salmonella species such as S. enteritidis or a lytic agent including the same, (ii) a bacteriophage having a broad host range against Salmonella species or a lytic agent including the same, (iii) a host-specific bacteriophage or an effective lytic agent for a Salmonella species including the same, or (iv) a bacteriophage or a lytic agent including the same, capable of effectively controlling S. typhimurium, particularly S. typhimurium having multidrug resistance. The present disclosure provides bacteriophages having specific genomic DNA sequences, lytic agents for Salmonella species including the same, and compositions including the same.

### SEQUENCE LISTING

This application contains a sequence listing in computer readable form (File name: PH-10243-PCT_Sequence Listing.xml; date of creation: Jul. 30, 2025; File size: 1,148,401 bytes) which is incorporated herein by reference in its entirety and forms part of the disclosure.

### TECHNICAL FIELD

One or more embodiments of the present invention relates to bacteriophages, lytic agents comprising a bacteriophage, compositions comprising the same, and a method for controlling a Salmonella species using the same.

### BACKGROUND

Salmonella species are one of the main causative organisms of food poisoning, and infect animals such as humans and livestock to cause salmonellosis such as diarrhea. Salmonella species are present in the digestive tracts of animals such as humans and livestock, and cause contamination when excreted in feces. Infection with Salmonella species is often caused by consumption of a food or beverage or feed contaminated with Salmonella species.

Conventionally, low molecular weight compounds have been used as antimicrobial agents against Salmonella species. However, since continuous use of these compounds causes negative effects such as emergence of multidrug resistant bacteria, new control measures have been explored. In recent years, bacteriophages have attracted attention as a novel means for controlling Salmonella species because they have high target specificity, do not damage the microbiota, and have low toxicity (Non Patent Literature 1).

The bacteriophage (often abbreviated herein simply as “phage”) is a generic term for viruses that infect only bacteria. Many phages adsorb to target bacteria serving as a host, then inject their own DNA into the bacteria, and self-amplify using the translation mechanism of the bacteria. Furthermore, by lysing the bacteria, the amplified phages are dispersed and repeatedly infect new target bacteria (Non Patent Literature 2).

Phages that exhibit lytic activity against Salmonella species have been reported in, for example, Patent Literatures 1 and 2. Phages that lyse Salmonella species can be used, for example, to control Salmonella species in poultry and pig farming, and to detect and control Salmonella species in the food industry field (Non Patent Literature 3). In fact, products containing phages that exhibit lytic activity against Salmonella species have already been marketed, such as BAFASAL® (Proteon Pharmaceuticals), which is a feed additive for preventing Salmonella species infection in chickens, and SalmoFresh™ (intralytix) and PhageGuard (Micreos), which are food-processing preparations for sterilizing Salmonella species in foods (Non Patent Literature 4).

### PATENT LITERATURE

PTL 1: WO2013-027146
PTL 2: Japanese Unexamined Patent Application Publication No. 2014-217336

### Non-Patent Literature

NPL 1: Jun-Hyun Oh et al., 2017, J. Microbiol. Biotechnol., 27(12), 2075-2088
NPL 2: Sharma S. et al., Folia Microbiol., 2017, 62: 17-55
NPL 3: Shuai Wei et al., Microorganisms, 2019, 7, 570
NPL 4: Katarzyna Zbikowska et al., Animals, 2020, 10, 872

### SUMMARY

As described above, phages that exhibit lytic activity against Salmonella species have been discovered, and commercial products using them have been launched. However, since it is assumed that when a specific phage is frequently used, Salmonella species having resistance to the phage may emerge, the discovery of novel phages is still required.

For example, since S. enteritidis is the serotype of Salmonella species most frequently detected in chickens (NPL 1), a phage that exhibits a wide range of lytic activity against strains of this serotype is desirable.

In addition, one of the characteristics required for phages used in lytic compositions is a broad host range for Salmonella species. A phage having a broad host range is desirable because it can be applied to various Salmonella species and broadens the range of application.

In addition, a lytic composition using a phage with high host specificity targeting specific Salmonella species is also desirable because it is useful for, for example, identifying the serotype of bacteria causing food poisoning.

In addition, among the Salmonella species, in particular, S. typhimurium has a problem of acquiring multidrug resistance, which is resistance to a plurality of antibacterial agents. Typical examples include bacterial strains having resistance to five drugs: ampicillin, chloramphenicol, streptomycin, sulfa drugs, and tetracycline. Such multidrug resistant S. typhimurium has been found to be widespread throughout the world, in part due to increased use of antibiotics in animal husbandry and in hospitals. Therefore, a technique capable of effectively controlling S. typhimurium has been desired.

Therefore, an object of the present disclosure is to provide (i) a novel bacteriophage having lytic activity against a Salmonella species such as S. enteritidis or a lytic agent comprising the same, (ii) a bacteriophage having a broad host range against Salmonella species or a lytic agent comprising the same, (iii) a host-specific bacteriophage or an effective lytic agent comprising the same for a Salmonella species, or (iv) a bacteriophage capable of effectively controlling S. typhimurium, particularly S. typhimurium having multidrug resistance, or a lytic agent comprising the same.

The present inventors have isolated novel phages from natural wastewater or soil, evaluated the lytic activity of the phages against various Salmonella species, and analyzed the genome sequences of the phages, using a technique for detecting lytic plaques formed on soft agar media on which Salmonella bacteria have been cultured.

As a result, it has been revealed that seven bacteriophages having specific genomic DNA sequences (corresponding to the first phage in the present specification) have lytic activity against a specific Salmonella species.

In addition, it has been revealed that three specific bacteriophages (corresponding to the second phage in the present specification) have broad lytic activity against Salmonella species, specifically lytic activity against S. enteritidis, S. typhimurium, S. infantis, S. Montevideo, and S. javiana.

In addition, it has been revealed that one specific bacteriophage (corresponding to the third phage in the present specification) has lytic activity against S. typhimurium, particularly against various S. typhimurium exhibiting multidrug resistance to antibiotics.

In addition, it has been revealed that a bacteriophage having a specific genomic DNA sequence (corresponding to the fourth phage in the present specification) has lytic activity against a specific Salmonella species.

In addition, it has been revealed that a bacteriophage having genomic DNA comprising a gene encoding an endonuclease consisting of a specific amino acid sequence (corresponding to the fifth phage in the present specification) has lytic activity against a specific Salmonella species.

In addition, it has been revealed that a specific bacteriophage (corresponding to the sixth phage in the present specification) is a novel bacteriophage having lytic activity against Salmonella species, specifically, lytic activity against S. enteritidis, S. typhimurium, and S. javiana.

In addition, it has been revealed that a bacteriophage having a specific genomic DNA sequence (corresponding to the seventh phage in the present specification) has lytic activity against a specific Salmonella species.

One or more embodiments of the present invention have been completed based on the above research and development results, and specifically provides the following embodiments.
[1] A bacteriophage that exhibits lytic activity against a Salmonella species, the bacteriophage having genomic DNA comprising a gene encoding a tail fiber protein consisting of an amino acid sequence of any one of (a) to (c) below and having recognition activity for target bacteria:
   (a) the amino acid sequence of SEQ ID NO: 18;
   (b) an amino acid sequence in which one or a plurality of amino acids are added, deleted, and/or substituted in the amino acid sequence of SEQ ID NO: 18; and
   (c) an amino acid sequence having 99% or more sequence identity to the amino acid sequence of SEQ ID NO: 18.
[2] The bacteriophage according to [1], wherein the gene encoding the tail fiber protein comprises a nucleotide sequence of any one of (d) to (f) below:
   (d) the nucleotide sequence of SEQ ID NO: 19;
   (e) a nucleotide sequence in which one or a plurality of nucleotides are added, deleted, and/or substituted in the nucleotide sequence of SEQ ID NO: 19; and
   (f) a nucleotide sequence having 97% or more sequence identity to the nucleotide sequence of SEQ ID NO: 19.
[3] The bacteriophage according to [1] or [2], wherein the sequence of the genomic DNA comprises a nucleotide sequence of any one of (g) to (k) below:
   (g) the nucleotide sequence of SEQ ID NO: 20;
   (h) a nucleotide sequence in which, in the nucleotide sequence of SEQ ID NO: 20, one or a plurality of nucleotides are added, deleted, and/or substituted in the nucleotide sequence other than the nucleotide sequence of the gene;
   (i) a nucleotide sequence having 90% or more sequence identity to the nucleotide sequence of SEQ ID NO: 20, in the nucleotide sequence other than the nucleotide sequence of the gene;
   (j) a nucleotide sequence in which one or a plurality of nucleotides are added, deleted, and/or substituted in the nucleotide sequence of SEQ ID NO: 20; and
   (k) a nucleotide sequence having 95% or more sequence identity to the nucleotide sequence of SEQ ID NO: 20.
[4] A lytic agent for S. enteritidis, comprising a bacteriophage having a genomic DNA sequence comprising a nucleotide sequence of any one of (a) to (c) below:
   (a) the nucleotide sequence of any one of SEQ ID NOs: 1 to 7;
   (b) a nucleotide sequence in which one or a plurality of nucleotides are added, deleted, and/or substituted in the nucleotide sequence of any one of SEQ ID NOs: 1 to 7; and
   (c) a nucleotide sequence having 99% or more sequence identity to the nucleotide sequence of any one of SEQ ID NOs: 1 to 7.
[5] A bacteriophage that exhibits lytic activity against a Salmonella species, the bacteriophage having a genomic DNA sequence comprising a nucleotide sequence of any one of (a) to (c) below:
   (a) the nucleotide sequence of SEQ ID NO: 13;
   (b) a nucleotide sequence in which one or a plurality of nucleotides are added, deleted, and/or substituted in the nucleotide sequence of SEQ ID NO: 13; and
   (c) a nucleotide sequence having 90% or more sequence identity to the nucleotide sequence of SEQ ID NO: 13.
[6] A lytic agent for a Salmonella species, comprising a bacteriophage having a genomic DNA sequence comprising a nucleotide sequence of any one of (a) to (c) below:
   (a) the nucleotide sequence of SEQ ID NO: 14;
   (b) a nucleotide sequence in which one or a plurality of nucleotides are added, deleted, and/or substituted in the nucleotide sequence of SEQ ID NO: 14; and
   (c) a nucleotide sequence having 95% or more sequence identity to the nucleotide sequence of SEQ ID NO: 14.
[7] A lytic agent for a Salmonella species, comprising a bacteriophage having genomic DNA comprising a gene encoding an endonuclease consisting of an amino acid sequence of any one of (a) to (c) below and having endonuclease activity:
   (a) the amino acid sequence of SEQ ID NO: 15;
   (b) an amino acid sequence in which one or a plurality of amino acids are added, deleted, and/or substituted in the amino acid sequence of SEQ ID NO: 15; and
   (c) an amino acid sequence having 90% or more sequence identity to the amino acid sequence of SEQ ID NO: 15.
[8] The lytic agent according to [7], wherein the gene encoding the endonuclease comprises a nucleotide sequence of any one of (d) to (f) below:
   (d) the nucleotide sequence of SEQ ID NO: 16;
   (e) a nucleotide sequence in which one or a plurality of nucleotides are added, deleted, and/or substituted in the nucleotide sequence of SEQ ID NO: 16; and
   (f) a nucleotide sequence having 90% or more sequence identity to the nucleotide sequence of SEQ ID NO: 16.
[9] The lytic agent according to [7] or [8], wherein the sequence of the genomic DNA comprises a nucleotide sequence of any one of (g) to (k) below:
   (g) the nucleotide sequence of SEQ ID NO: 17;
   (h) a nucleotide sequence in which, in the nucleotide sequence of SEQ ID NO: 17, one or a plurality of nucleotides are added, deleted, and/or substituted in the nucleotide sequence other than the nucleotide sequence of the gene;
   (i) a nucleotide sequence having 80% or more sequence identity to the nucleotide sequence of SEQ ID NO: 17, in the nucleotide sequence other than the nucleotide sequence of the gene;
   (j) a nucleotide sequence in which one or a plurality of nucleotides are added, deleted, and/or substituted in the nucleotide sequence of SEQ ID NO: 17; and
   (k) a nucleotide sequence having 90% or more sequence identity to the nucleotide sequence of SEQ ID NO: 17.
[10] A lytic agent for S. enteritidis, comprising a bacteriophage having genomic DNA comprising a gene encoding a tail spike protein consisting of the amino acid sequence of SEQ ID NO: 21, wherein the sequence of the genomic DNA comprises a nucleotide sequence of any one of (a) to (e) below:
   (a) the nucleotide sequence of SEQ ID NO: 23;
   (b) a nucleotide sequence in which, in the nucleotide sequence of SEQ ID NO: 23, one or a plurality of nucleotides are added, deleted, and/or substituted in the nucleotide sequence other than the nucleotide sequence of the gene;
   (c) a nucleotide sequence having 99% or more sequence identity to the nucleotide sequence of SEQ ID NO: 23, in the nucleotide sequence other than the nucleotide sequence of the gene;
   (d) a nucleotide sequence in which one or a plurality of nucleotides are added, deleted, and/or substituted in the nucleotide sequence of SEQ ID NO: 23; and
   (e) a nucleotide sequence having 99% or more sequence identity to the nucleotide sequence of SEQ ID NO: 23.
[11] The lytic agent according to [10], wherein the gene encoding the tail spike protein comprises the nucleotide sequence of SEQ ID NO: 22.
[12] The bacteriophage according to any one of [1] to [3], wherein the Salmonella species is S. enteritidis, S. typhimurium, and S. javiana;
   the bacteriophage according to [5], wherein the Salmonella species is S. typhimurium;
   the lytic agent according to [6], wherein the Salmonella species is S. Montevideo; or
   the lytic agent according to any one of [7] to [9], wherein the Salmonella species is S. typhimurium.
[13] A composition comprising the bacteriophage according to any one of [1] to [3], the lytic agent according to [4], the bacteriophage according to [5], the lytic agent according to [6], the lytic agent according to any one of [7] to [9], the lytic agent according to [10] or [11], or the bacteriophage or lytic agent according to [12].
[14] A composition for controlling S. enteritidis, S. typhimurium, and S. javiana, comprising the bacteriophage according to any one of [1] to [3];
   a composition for controlling S. enteritidis, comprising the lytic agent according to [4];
   a composition for controlling S. typhimurium, comprising the bacteriophage according to [5];
   a composition for controlling a Salmonella species or a composition for controlling S. Montevideo, comprising the lytic agent according to [6];
a composition for controlling a Salmonella species or a composition for controlling S. typhimurium, comprising the lytic agent according to any one of [7] to [9]; or
a composition for controlling S. enteritidis, comprising the lytic agent according to [10] or [11].
[15] The composition according to [13] or [14], which is a pharmaceutical composition.
[16] The composition according to [13] or [14], which is a food or beverage additive, a feed additive or a drinking water additive.
[17] The composition according to [13] or [14], which is a food or beverage product or feed.
[18] The composition according to [13] or [14], which is a cleaning agent, a disinfectant, a sterilizing agent, or an antibacterial agent.
[19] The composition according to any one of [13] to [18], further comprising another bacteriophage that exhibits lytic activity against a Salmonella species.
[20] A method for controlling a Salmonella species, the method comprising a contact step of bringing the bacteriophage according to any one of [1] to [3], the lytic agent according to [4], the bacteriophage according to [5], the lytic agent according to [6], the lytic agent according to any one of [7] to [9], the lytic agent according to [10] or [11], the bacteriophage or lytic agent according to [12], or the composition according to any one of [13] to [19] into contact with an application target.
[21] A method for treating or preventing an infectious disease caused by a Salmonella species in a target, the method comprising an administration step of administering to the target the bacteriophage according to any one of [1] to [3], the lytic agent according to [4], the bacteriophage according to [5], the lytic agent according to [6], the lytic agent according to any one of [7] to [9], the lytic agent according to [10] or [11], the bacteriophage or lytic agent according to [12], or the composition according to any one of [13] to [19].
[22] A method for identifying a Salmonella species, the method comprising:
   a culturing step of culturing test bacteria isolated from a specimen suspected of containing a Salmonella species to obtain a culture;
   a mixing step of mixing the culture with the bacteriophage according to any one of [1] to [3], the lytic agent according to [4], the bacteriophage according to [5], the lytic agent according to [6], the lytic agent according to any one of [7] to [9], the lytic agent according to [10] or [11], the bacteriophage or lytic agent according to [12], or the composition according to any one of [13] to [19] to obtain a mixture;
   a mixture culturing step of culturing the mixture under predetermined conditions; and
   a determination step of determining that the test bacteria are a Salmonella species when the test bacteria are lysed after the mixture culturing step.
[23] The method according to [22], wherein in the mixture culturing step, the mixture further comprises a soft agar-containing liquid medium, and the mixture is cultured on a solid medium.
[24] The method according to [22], wherein in the culturing step, the culture comprises a soft agar-containing liquid medium, and the culture is cultured on a solid medium.
[25] The method according to [22], further comprising an isolation step of isolating the test bacteria from the specimen suspected of containing a Salmonella species before the culturing step.

The present specification incorporates the disclosure contents of Japanese Patent Application No. 2023-057024, No. 2023-057035, No. 2023-057554, No. 2023-057617, No. 2023-057261, No. 2023-057268, No. 2023-057565, and No. 2023-057568, which form the basis for the priority of the present application.

### Advantageous Effects of Invention

One or more embodiments of the present invention can provide a novel bacteriophage or a lytic agent having lytic activity against a Salmonella species, or a composition comprising the foregoing. One or more embodiments of the present invention can also provide a bacteriophage or a lytic agent having a broad host range for Salmonella species, or a composition comprising the foregoing. One or more embodiments of the present invention can also provide a bacteriophage or a lytic agent that can lyse a specific target Salmonella species or a composition comprising the foregoing. One or more embodiments of the present invention can also provide a bacteriophage or a lytic agent that can effectively control S. typhimurium, particularly S. typhimurium with multidrug resistance, or a composition comprising the foregoing.

### BRIEF DESCRIPTION OF THE DRAWINGS

FIG. 1A shows the lytic activity of the first bacteriophage obtained in Example 1. Shown is a photograph of agar plates after culturing when Salmonella species were spread on the agar plates, the first phage purified solution was added dropwise, and culturing under static conditions was performed.

FIG. 1B is a plate diagram corresponding to FIG. 1A, and shows the strain IDs of the Salmonella species spread on the plates and the positions of the phage purified solution added dropwise. Here, a indicates the positions of the purified solution of the phage having the genomic DNA sequence of SEQ ID NO: 7.

FIG. 2A shows the lytic activity of the first bacteriophage obtained in Example 1. Shown is a photograph of agar plates after culturing when Salmonella species were spread on the agar plates, the first phage purified solutions were added dropwise, and culturing under static conditions was performed.

FIG. 2B is a plate diagram corresponding to FIG. 2A, and shows the strain IDs of the Salmonella species spread on the plates and the positions of the phage purified solutions added dropwise. Here, a, b, c, d, e, f, and g indicate the positions of the purified solutions of the phages having the genomic DNA sequences of SEQ ID NOs: 1, 2, 3, 4, 5, 6, and 7, respectively.

FIG. 3A shows the lytic activity of the second bacteriophage obtained in Example 2. Shown is a photograph of agar plates after culturing when Salmonella species were spread on the agar plates, the second phage purified solution was added dropwise, and culturing under static conditions was performed.

FIG. 3B is a plate diagram corresponding to FIG. 3A, and shows the strain IDs of the Salmonella species spread on the plates and the positions of the phage purified solution added dropwise. Here, a indicates the positions of the purified solution of the phage having the genomic DNA sequence of SEQ ID NO: 10.

FIG. 4A shows the lytic activity of the second bacteriophage obtained in Example 2, following FIGS. 3A and 3B.

FIG. 4B shows is a plate diagram corresponding to FIG. 4A.

FIG. 5A shows the lytic activity of the second bacteriophage obtained in Example 2. Shown is a photograph of agar plates after culturing when Salmonella species were spread on the agar plates, the second phage purified solutions were added dropwise, and culturing under static conditions was performed.

FIG. 5B is a plate diagram corresponding to FIG. 5A, and shows the strain IDs of the Salmonella species spread on the plates and the positions of the phage purified solutions added dropwise. Here, a, b, and c indicate the positions of the purified solutions of the phages having genomic DNA sequences of SEQ ID NOs: 10, 11, and 12, respectively.

FIG. 6A shows the lytic activity of the third bacteriophage obtained in Example 3. Shown is a photograph of agar plates after culturing when Salmonella species (S. typhimurium) were spread on the agar plates, the third phage purified solution was added dropwise, and culturing under static conditions was performed.

FIG. 6B is a plate diagram corresponding to FIG. 6A, and shows the strain IDs of the Salmonella species (S. typhimurium) spread on the plates and the positions of the phage purified solution added dropwise. Here, a indicates the positions of the purified solution of the phage having the genomic DNA sequence of SEQ ID NO: 13.

FIG. 7A shows the lytic activity of the fourth bacteriophage obtained in Example 4. Shown is a photograph of agar plates after culturing when Salmonella species were spread on the agar plates, and the fourth phage purified solution was added dropwise, and culturing under static conditions was performed.

FIG. 7B is a plate diagram corresponding to FIG. 7A, and shows the strain IDs of the Salmonella species spread on the plates and the positions of the phage purified solution added dropwise. Here, a indicates the positions of the purified solution of the phage having the genomic DNA sequence of SEQ ID NO: 14.

FIG. 8A shows the lytic activity of the fifth bacteriophage obtained in Example 5. Shown is a photograph of agar plates after culturing when Salmonella species were spread on the agar plates, the fifth phage purified solution was added dropwise, and culturing under static conditions was performed.

FIG. 8B is a plate diagram corresponding to FIG. 8A, and shows the strain IDs of the Salmonella species spread on the plates and the positions of the phage purified solution added dropwise. Here, a indicates the positions of the purified solution of the phage having the genomic DNA sequence of SEQ ID NO: 17.

FIG. 9A shows the lytic activity of the sixth bacteriophage obtained in Example 6. Shown is a photograph of agar plates after culturing when Salmonella species were spread on the agar plates, the sixth phage purified solution was added dropwise, and culturing under static conditions was performed.

FIG. 9B is a plate diagram corresponding to FIG. 9A, and shows the strain IDs of the Salmonella species spread on the plates and the positions of the phage purified solution added dropwise. Here, a indicates the positions of the purified solution of the phage having the genomic DNA sequence of SEQ ID NO: 20.

FIG. 10A shows the lytic activity of the seventh bacteriophage obtained in Example 7. Shown is a photograph of agar plates after culturing when Salmonella species were spread on the agar plates, the seventh phage purified solution was added dropwise, and culturing under static conditions was performed.

FIG. 10B is a plate diagram corresponding to FIG. 10A, and shows the strain IDs of the Salmonella species spread on the plates and the positions of the phage purified solution added dropwise. Here, a indicates the positions of the purified solution of the phage having the genomic DNA sequence of SEQ ID NO: 23.

FIG. 11 shows the alignment between the query sequence (the amino acid sequence of the tail tip protein of the obtained second phage (SEQ ID NO: 8)) and the searched sequence in Example 2.

FIG. 12A shows the multiple alignment performed in Example 6.

FIG. 12B shows, following FIG. 12A, the multiple alignment performed in Example 6.

FIG. 12C shows, following FIG. 12B, the multiple alignment performed in Example 6.

### DETAILED DESCRIPTION

One or more embodiments of the present invention are described in detail below.

### Definitions

The terms used herein are defined below.

As used herein, the term “lysis” refers to a phenomenon in which the cell membranes of bacteria are destroyed. By lysis, the bacteria are killed. Lysis starts with phages specifically adsorbing to the target bacteria and injecting their DNA into the cells of the target bacteria via the tails. Thereafter, they use the bacterial translation mechanism to replicate themselves and produce a large number of progeny phages, and then lyse the bacteria to release the progeny phages to the outside world.

As used herein, the term “lytic agent” refers to an agent comprising a bacteriophage having lytic activity against the target bacteria. The lytic agent can be a lytic agent for specifically lysing the target bacteria (target bacteria-specific lytic agent). The lytic agent may be the bacteriophage itself.

As used herein, the term “bacteria” refers to one of the major lineages of organisms. Together with archaea and eukaryotes, it constitutes the three domains of life that make up the entire biological world. Bacteria consist of cells without a cell nucleus and are capable of self-replication if there is a nutrient source.

As used herein, the term “target bacteria” refers to host bacteria which can be a target of the phage constituting a lytic agent of one or more embodiments of the present invention or the phage included in a composition of one or more embodiments of the present invention. Specifically, they are, for example, bacteria having, on the cell outer membranes, membrane surface receptors that are recognized by the phage. Alternatively, they are, for example, bacteria having, on the cell outer membranes, membrane surface receptors that are recognized by a tail fiber protein, a tail tip protein, a tail spike protein, or a tail tube protein consisting of a specific amino acid sequence. The “membrane surface receptor” is a site to which, for example, the tail and tail fibers of a phage bind, and is composed of protein, lipopolysaccharide, pili, or the like present in the outer layer of the bacterial outer membrane. The target bacteria herein are particularly Salmonella species.

As used herein, the term “Salmonella species” refers to bacteria belonging to the genus Salmonella. Salmonella species are classified into two species: Salmonella enterica and Salmonella bongori, and the former is further divided into six subspecies: ssp. enterica, ssp. salamae, ssp. arizonae, ssp. diarizonae, ssp. houtenae, and ssp. indica. Salmonella species are also serotyped on the basis of two surface structures: somatic antigen (also referred to as O antigen) and flagellar antigen (also referred to as H antigen). The subspecies name and serotype of Salmonella species are indicated by adding subspecies (ssp.) and serover (or serotype), respectively, after the bacterial name. The names of Salmonella species are sometimes abbreviated with the serotype described after S. For example, S. enterica ssp. enterica serovar typhimurium may be abbreviated as S. typhimurium. The smallest unit of classification is a strain, which refers to a population of cells that is considered genetically homogeneous.

Specific examples of serotypes of Salmonella species include S. enteritidis (Salmonella enterica ssp. enterica serovar enteritidis), S. typhimurium (Salmonella enterica ssp. enterica serovar typhimurium), S. Newport, S. I 4, [5], 12:i:-, S. javiana (Salmonella enterica ssp. enterica serovar javiana), S. Heidelberg, S. infantis (Salmonella enterica ssp. enterica serovar infantis), S. Saintpaul, S. Muenchen, S. Montevideo (Salmonella enterica ssp. enterica serovar Montevideo), S. Braenderup, S. Oranienburg, S. Thompson, S. Mississippi, S. Agona, S. Typhi, S. Bareilly, S. Paratyphi B, S. Poona, S. Berta, S. Abony, S. Anatum, S. Baildon, S. Bredeney, S. Chester, S. Gaminara, S. Hartford, S. Kentucky, S. Kiambu, S. Mbandaka, S. Nchanga, S. Reading, S. Senftenberg, S. Stanley, S. Virchow, S. Urbana, etc.

As used herein, the term “lytic agent for a Salmonella species” refers to a lytic agent for lysing a Salmonella species. Similarly, the term “lytic agent for S. enteritidis” refers to a lytic agent for lysing S. enteritidis. The lytic agent for S. enteritidis may be a lytic agent for specifically lysing S. enteritidis (S. enteritidis-specific lytic agent). The term “lytic agent for S. Montevideo” refers to a lytic agent for lysing S. Montevideo. The lytic agent for S. Montevideo may be a lytic agent for specifically lysing S. Montevideo (S. Montevideo-specific lytic agent). The term “lytic agent for S. typhimurium” refers to a lytic agent for lysing S. typhimurium. The lytic agent for S. typhimurium may be a lytic agent for specifically lysing S. typhimurium (S. typhimurium-specific lytic agent).

As used herein, “control” of bacteria means killing the bacteria and/or inhibiting the growth of the bacteria.

As used herein, “multidrug resistance” means exhibiting resistance to a plurality of antibacterial agents (e.g., antibiotics). Examples of the antibacterial agents include, but are not particularly limited to, ampicillin, chloramphenicol, streptomycin, sulfa drugs, tetracycline, kanamycin, sulfamethoxazole/trimethoprim, cefazolin, cefotaxime, nalidixic acid, and gentamicin.

As used herein, “bacteriophage” (as described above, often simply abbreviated as “phage” in the present specification) is a general term for viruses that infect bacteria. A general phage is composed of three parts: the head, the tail, and the tail fibers. The head is composed of a capsid (virus shell) having an icosahedral structure and composed of capsomeres which are outer coat proteins, and contains the phage genomic DNA in its inner space. The tail has a tubular structure composed of tail tube proteins and sheath proteins covering them. One end of the tail is connected to the head and the other end to the tail fibers. The tail serves as an introduction tube through which the genomic DNA from the head is injected into a host bacterial cell. The tail fibers are composed of several fiber structures composed of tail fiber proteins. The tail and tail fibers are responsible for the host recognition function and the adsorption function of recognizing receptors present on the outer membrane surface of the host bacteria and adsorbing to the cell surface. Phages have extremely high host specificity, which is based on the functions of the tail and tail fibers. More specifically, any protein of the tail fiber protein, the tail tube protein, the tail tip protein, and the tail spike protein described below plays a central role in the functions.

In the present specification, “tail fiber protein” refers to a protein that constitutes the tail fibers of a phage, as described above. The tail fiber protein is known to play an important role in the specificity of the host recognition and adsorption ability of tails and tail fibers (Nobrega F. L. et al., Nat. Rev. Microbiol., 2018, 16: 760-773). Therefore, novel phages characterized by tail fiber proteins have very high utility value, as they can exhibit, for example, lytic activity even against bacteria that have, for example, infection resistance to known phages, since they have different host recognition sites even when the host bacteria are the same as those of known phages.

As used herein, the term “tail fiber gene” refers to a gene that is contained in the phage genomic DNA and encodes the tail fiber protein.

As used herein, the term “tail tube protein” refers to a protein that constitutes the tubular structure of the phage tail, as described above. Tail tube proteins are known to interact with tail fibers and play an important role in the specificity of host recognition and adsorption ability together with tail fibers (Maozhi Hu, et al., 2020, 9: 1, 855-867). As tail tube proteins, tail tube protein A and tail tube protein B are known. “Tail tube protein A” is a protein that forms a ring in a lower portion of the tubular structure of the tail and interacts with the tail fibers. “Tail tube protein B” is a protein that forms the lower end of the tubular structure of the tail and binds to a receptor present on the outer membrane surface of the host bacteria.

As used herein, the term “tail tube gene” refers to a gene that is contained in the genomic DNA of a phage and encodes the tail tube proteins. The term “tail tube protein A gene” refers to a gene encoding tail tube protein A, and the term “tail tube protein B gene” refers to a gene encoding tail tube protein B.

As used herein, the term “tail tip protein” refers to a protein constituting the tip of the tail of a phage, which plays a role in penetrating the cell walls of host bacteria due to its sharp structure, and also has a function of binding to a receptor of the host bacteria as described above. It is known that the tail tip protein has a function of binding to a receptor of host bacteria and thus plays an important role in host recognition and adsorption ability (Nobrega F. L. et al., Nat. Rev. Microbiol., 2018, 16: 760-773).

As used herein, the term “tail tip gene” refers to a gene that is contained in the genomic DNA of a phage and encodes the tail tip protein.

As used herein, the term “tail spike protein” refers to a protein constituting the tail tip of a phage, and has a function of binding to a receptor of host bacteria as described above. The tail spike protein forms, when a plate-like structure (tail plate) is present at the tail tip of the phage, a spike-like structure at the bottom of the plate. It is known that the tail spike protein plays an important role in host recognition and adsorption ability due to its function of binding to a receptor of host bacteria (Nobrega F. L. et al., Nat. Rev. Microbiol., 2018, 16: 760-773).

As used herein, the term “tail spike gene” refers to a gene that is contained in the genomic DNA of a phage and encodes the tail spike protein.

Note that the phages do not necessarily have all of the above-described tail fiber gene, tail tube gene, tail tip gene, and tail spike gene. The phages can comprise one, two, three, or all four of the tail fiber gene, the tail tube gene, the tail tip gene, and the tail spike gene.

As used herein, the term “endonuclease” refers to an enzyme that cleaves a polynucleotide chain within the polynucleotide chain. It is known that lytic phages take over the life-supporting mechanisms of host bacteria by various means upon infection, allowing only their self-replication while shutting down the replication of the host genome. Although the details of this mechanism have not yet been fully elucidated, it has long been established that degradation of the host genome by phage-derived nucleases is involved (Warren et al., Journal of Virology, Vol. 2, No. 4, 1968). Therefore, endonucleases from lytic phages are considered to be involved in the mechanism that shuts down the replication of the host genome.

Since phages do not infect eukaryotic organisms, preparations using phages are harmless to humans, animals, and plants. Note that the life cycle of phages is broadly classified into a “lytic cycle”, a “lysogenic cycle”, and a “lytic/lysogenic cycle”. In the lysogenic cycle, the phages integrate their DNA into the bacterial chromosome without lysing the target bacteria, and multiply along with the growth of the bacteria. On the other hand, in the lytic cycle, the phages self-replicate in the cells of the host bacteria, and then lyse the host bacteria to release a large number of progeny phages. Phages of one or more embodiments of the present invention can be phages that undergo the lytic cycle or the lytic/lysogenic cycle.

As used herein, “a plurality of” refers to 2 to 10, for example, 2 to 7, 2 to 5, 2 to 4, or 2 to 3.

As used herein, the term “nucleotide sequence identity” refers to a numerical value representing the proportion of positions where the types of nucleotides are identical within a comparison region between two nucleotide sequences. Nucleotide sequence identity can be calculated by aligning two nucleotide sequences such that the degree of nucleotide identity within the comparison region is maximized, even when the two nucleotide sequences have different lengths. A representative, non-limiting algorithm for performing such an analysis is BLAST. BLAST can be used in various software and Web services. For example, nucleotide sequence identity can be easily calculated using genetic information processing software GENETYX (https://www.genetyx.co.jp/), BLAST server provided by NCBI (https://blast.ncbi.nlm.nih.gov/Blast.cgi), etc. In addition to BLAST, there are other algorithms such as FASTA, which can be used if reasonable identity can be calculated. It is also possible to perform nucleotide sequence identity analysis using analysis algorithms such as MUMmer. Note that depending on the software or analysis server, sequence identity may be indicated by indexes such as Average Nucleotide Identity (ANI), which can also be used. Note that when aligning long nucleotide sequences such as phage genomic DNA using the above software or web services, the comparison region may be automatically determined, and sequence identity within that comparison region may be calculated. Therefore, the above sequence identity may be present in the region automatically aligned by the above software or web services. For example, in analysis using the BLAST server provided by NCBI, Query sequence and Subject sequence are automatically aligned in the maximum alignable region to determine a comparison region, sequence identity in the comparison region is calculated, and the ratio of the comparison region to the entire region of the Query sequence is calculated as a value called Query Cover in some cases. In such a case, the sequence identity in the entire region of the aligned nucleotide sequences can be estimated based on the result. For example, the value obtained by multiplying the Query Cover value by the value of the sequence identity in the comparison region can be used as the estimated value of the sequence identity in the entire region. At this time, in order to increase the accuracy of the estimated value, for example, a further correction may be applied, such as, in the calculation, additionally using the sequence identity expected in the region other than the alignment region. Note that when the phage genomic DNA is packaged, it is in a linear form or a circular form. In next-generation genome sequencer analysis, genomic DNA is fragmented, then the nucleotide sequences of the fragments are read, and the sequence is determined through an analysis that assembles the reads. In the case of phages, they are often assembled without specifying a reference genomic DNA sequence (de novo assembly). Therefore, it is difficult to unambiguously determine the starting points and ends of analyzed genomes (Merrill, B. D., et al. BMC Genomics, 2016 17, 679). Therefore, the start points and ends of the genome sequences to be compared may be different, and are automatically considered in the analysis using software or analysis servers.

As used herein, “highly stringent conditions” refers to environmental conditions under which non-specific hybridization is unlikely to occur. Under highly stringent conditions, hybrids can be formed with nucleic acids having a target nucleotide sequence, but hybrids substantially cannot be formed with nucleic acids having non-specific nucleotide sequences. In general, highly stringent conditions refer to conditions of low salt concentration and high temperature. The low salt concentration refers to, for example, 15 to 750 mM, particularly 15 to 500 mM, 15 to 300 mM or 15 to 200 mM. The high temperature refers to, for example, 50 to 68° C., or 55 to 70° C. As a specific example of highly stringent conditions, washing at 65° C. using 0.1×SSC and 0.1% SDS in washing after hybridization can be mentioned.

As used herein, “amino acid sequence identity” is a numerical value representing the proportion of positions where the types of amino acid residues are identical within a comparison region between two amino acid sequences. Amino acid sequence identity can be calculated by aligning two amino acid sequences such that the degree of amino acid identity within the comparison region is maximized even when the two amino acid sequences have different lengths. A representative, non-limiting algorithm for performing such an analysis is BLAST. BLAST can be used in various software and Web services. For example, amino acid sequence identity can be easily calculated using genetic information processing software GENETYX (https://www.genetyx.co.jp/), BLAST server provided by NCBI (https://blast.ncbi.nlm.nih.gov/Blast.cgi), etc. In addition to BLAST, there are other algorithms such as FASTA, which can be used if reasonable identity can be calculated.

In the present specification, “(amino acid) substitution” particularly refers to substitution within conservative amino acid groups having similar properties in terms of charge, side chain, polarity, aromaticity, or the like among the 20 amino acids constituting natural proteins. Examples include substitutions within the uncharged polar amino acid group having a low-polarity side chain (Gly, Asn, Gln, Ser, Thr, Cys, and Tyr), the branched amino acid group (Leu, Val, and Ile), the neutral amino acid group (Gly, Ile, Val, Leu, Ala, Met, and Pro), the neutral amino acid group having a hydrophilic side chain (Asn, Gln, Thr, Ser, Tyr, and Cys), the acidic amino acid group (Asp and Glu), the basic amino acid group (Arg, Lys, and His), or the aromatic amino acid group (Phe, Tyr, and Trp). Substitutions may be present singly or in combinations of two or more. Amino acid substitutions within these groups are particularly used because they are known to be less likely to cause changes in the properties of the polypeptides.

### 1. Bacteriophage/Lytic Agent

A first aspect of one or more embodiments of the present invention is the following bacteriophage which exhibits lytic activity against Salmonella species and a lytic agent comprising the same.

### <First Phage/Lytic Agent>

### Overview

One or more embodiments of the present invention provides a phage having lytic activity against Salmonella species and having a feature below (which may be referred to as “first phage” in the present specification), and a lytic agent comprising the phage (which may be referred to as “first lytic agent” in the present specification). The first lytic agent is a lytic agent which particularly exhibits specific lytic activity against a specific Salmonella species, for example, a lytic agent for S. enteritidis. The first lytic agent comprises a bacteriophage having a genomic DNA sequence comprising a specific nucleotide sequence.

According to the first lytic agent, the target bacteria can be lysed and controlled.

### (Feature)

The first lytic agent is in particular a lytic agent for a Salmonella species, in particular a lytic agent for S. enteritidis. The first lytic agent comprises the first phage having lytic activity against a Salmonella species and having the following feature.

The first phage has a genomic DNA sequence comprising a specific nucleotide sequence.

The present inventors have discovered seven types of phages having lytic activity specifically against bacterial strains of S. enteritidis and found that the genomic DNA sequences of these phages (SEQ ID NOs: 1 to 7, respectively) have extremely high sequence identity. For example, when the sequence identity (Identity) of the shortest genomic DNA sequence of SEQ ID NO: 7 to the genomic DNA sequences of SEQ ID NOs: 1 to 6 was calculated using genetic information processing software GENETYX (https://www.genetyx.co.jp/), it was 100% across the entire region.

The first phage has a genomic DNA sequence comprising or consisting of a nucleotide sequence of any one of the following (a) to (c):
(a) the nucleotide sequence of any one of SEQ ID NOs: 1 to 7;
(b) a nucleotide sequence in which one or a plurality of nucleotides are added, deleted, and/or substituted in the nucleotide sequence of any one of SEQ ID NOs: 1 to 7; and
(c) a nucleotide sequence having 99% or more sequence identity to the nucleotide sequence of any one of SEQ ID NOs: 1 to 7.

In particular, the sequence identity defined in (c) is 99.1% or more, 99.2% or more, 99.3% or more, 99.4% or more, 99.5% or more, 99.6% or more, 99.7% or more, 99.8% or more, or 99.9% or more.

The first phage is useful for treating or preventing food poisoning because it can particularly exhibit broad lytic activity against S. enteritidis, which is the most frequently detected serotype in food poisoning in humans. The first phage can also exhibit lytic activity specifically against S. enteritidis, and thus is useful, for example, for identifying the serotype of bacteria responsible for food poisoning.

### <Second Phage/Lytic Agent>

### (Overview)

One or more embodiments of the present invention provides a phage having lytic activity against Salmonella species and having a feature below (sometimes referred to as “second phage” in the present specification), and a lytic agent comprising the phage (sometimes referred to as “second lytic agent” in the present specification). The second phage can exhibit lytic activity against S. enteritidis, S. typhimurium, S. infantis, S. Montevideo, and S. javiana. The second phage has a genomic DNA sequence comprising a gene encoding a tail tip protein consisting of a specific amino acid sequence.

The second phage can lyse and control Salmonella species serving as target bacteria.

### (Feature)

The second phage is a phage having lytic activity against Salmonella species and having the following feature.

The second phage has genomic DNA comprising a gene encoding a tail tip protein consisting of a specific amino acid sequence and having recognition activity for target bacteria.

The present inventors have discovered three types of phages having lytic activity against Salmonella species, and identified a tail tip protein (SEQ ID NO: 8) and a tail tip gene (SEQ ID NO: 9) from the genomic DNA sequences (SEQ ID NOs: 10 to 12, respectively) of these phages. The sequence identity of the genome sequences of the three types of phages is 99%, and the amino acid sequences of their respective tail tip proteins were as shown in SEQ ID NO: 8, and are completely identical to each other.

Such a tail tip protein consists of the amino acid sequence of SEQ ID NO: 8, which consists of 637 amino acid residues. In one or more embodiments of the present invention, the tail tip protein consisting of the amino acid sequence of SEQ ID NO: 8 can realize extremely useful host specificity that is specific to Salmonella species and exhibits a wide range of lytic activity against various bacterial species within the Salmonella genus.

### (1) Tail Tip Protein

The tail tip protein in the second phage consists of an amino acid sequence of any one of the following (a) to (c):
(a) the amino acid sequence of SEQ ID NO: 8;
(b) an amino acid sequence in which one or a plurality of amino acids are added, deleted, and/or substituted in the amino acid sequence of SEQ ID NO: 8; and
(c) an amino acid sequence having 99% or more sequence identity to the amino acid sequence of SEQ ID NO: 8.

In particular, the sequence identity defined in (c) is 99.1% or more, 99.2% or more, 99.3% or more, 99.4% or more, 99.5% or more, 99.6% or more, 99.7% or more, 99.8% or more, or 99.9% or more.

In particular, in the amino acid sequence defined in (b) or (c), the amino acid at the position corresponding to position 258 of SEQ ID NO: 8 of the tail tip protein is phenylalanine, and/or the amino acid at the position corresponding to position 617 of SEQ ID NO: 8 is serine. Note that such positions are numbered with the initiating methionine assigned as position 1.

### (2) Tail Tip Gene

The gene encoding the tail tip protein comprises, for example, a nucleotide sequence of any one of the following (d) to (f):
(d) the nucleotide sequence of SEQ ID NO: 9;
(e) a nucleotide sequence in which one or a plurality of nucleotides are added, deleted, and/or substituted in the nucleotide sequence of SEQ ID NO: 9; and
(f) a nucleotide sequence having 95% or more sequence identity to the nucleotide sequence of SEQ ID NO: 9.

Alternatively, a nucleotide sequence that hybridizes under highly stringent conditions to a nucleotide sequence complementary to the nucleotide sequence of SEQ ID NO: 9 can also be mentioned.

In particular, the sequence identity defined in (f) is 96% or more, 97% or more, 98% or more, or 99% or more.

### (3) Genomic DNA

The second phage has genomic DNA comprising a gene encoding the tail tip protein.

The genomic DNA sequence comprises or consists of, for example, a nucleotide sequence of any one of the following (g) to (k):
(g) the nucleotide sequence of any one of SEQ ID NOs: 10 to 12;
(h) a nucleotide sequence in which one or a plurality of nucleotides are added, deleted, and/or substituted in the nucleotide sequence other than the nucleotide sequence of the gene in the nucleotide sequence of any one of SEQ ID NOs: 10 to 12;
(i) a nucleotide sequence having 80% or more sequence identity to the nucleotide sequence of any one of SEQ ID NOs: 10 to 12, in the nucleotide sequence other than the nucleotide sequence of the gene;
(j) a nucleotide sequence in which one or a plurality of nucleotides are added, deleted, and/or substituted in the nucleotide sequence of any one of SEQ ID NOs: 10 to 12; and
(k) a nucleotide sequence having 90% or more sequence identity to the nucleotide sequence of any one of SEQ ID NOs: 10 to 12.

In particular, the sequence identity defined in (i) is 81% or more, 82% or more, 83% or more, 84% or more, 85% or more, 86% or more, 87% or more, 88% or more, 89% or more, 90% or more, 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

The nucleotide sequence defined in (i) is, in other words, a nucleotide sequence in which the nucleotide sequence other than a gene corresponding to the above-described gene has a sequence identity of 80% or more to the nucleotide sequence other than the nucleotide sequence of the above-described gene in the nucleotide sequence of any one of SEQ ID NOs: 10 to 12.

In particular, the sequence identity defined in (k) is preferably 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

In one or more embodiments, the second phage is characterized by having a genomic DNA sequence comprising a specific nucleotide sequence, and exhibits lytic activity against target bacteria. The genomic DNA sequence of the second phage may be a genomic DNA sequence comprising the nucleotide sequence of any one of SEQ ID NOs: 10 to 12 (113946 bp, 113936 bp, and 113949 bp, respectively), a nucleotide sequence in which one or a plurality of nucleotides are added, deleted, and/or substituted in the nucleotide sequence of any one of SEQ ID NOs: 10 to 12, or a nucleotide sequence having 80% or more, 81% or more, 82% or more, 83% or more, 84% or more, 85% or more, 86% or more, 87% or more, 88% or more, 89% or more, 90% or more, 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more sequence identity to the nucleotide sequence of any one of SEQ ID NOs: 10 to 12.

### (4) Advantages

Since the second phage can exhibit a wide range of lytic activity against various bacterial species of the genus Salmonella, it can effectively control Salmonella species. The second phage is also useful for treating or preventing food poisoning. Because the second phage has a broad host range, it can effectively cover the diversity of the target bacteria. Therefore, the second phage, which is a phage that exhibits a wide range of lytic activity against various bacterial species, is extremely useful.

### <Third Phage/Lytic Agent>

### (Overview)

One or more embodiments of the present invention provides a phage having lytic activity against Salmonella species and having a feature below (sometimes referred to as “third phage” in the present specification), and a lytic agent comprising the phage (sometimes referred to as “third lytic agent” in the present specification). The third phage exhibits lytic activity against at least one bacterium selected from the group consisting of S. typhimurium, and particularly exhibits lytic activity against S. typhimurium having multidrug resistance. The third phage has a specific genomic DNA sequence.

Note that the target bacteria of the third phage are not limited to S. typhimurium. The third phage can effectively control S. typhimurium, particularly S. typhimurium having multidrug resistance, but may also exert a control effect on other serotypes serving as the target bacteria such as S. enteritidis, S. infantis or S. javiana.

According to the third phage, S. typhimurium serving as the target bacteria, particularly S. typhimurium having multidrug resistance, can be effectively lysed and controlled.

### (Feature)

The third phage is a phage having lytic activity against Salmonella species and having the following feature.

The third phage has genomic DNA comprising a specific nucleotide sequence.

The present inventors have discovered one type of phage having lytic activity against Salmonella species, and identified the genomic DNA sequence of this phage (SEQ ID NO: 13).

The third phage is highly useful because it is specific to Salmonella species and exhibits a wide range of lytic activity against S. typhimurium, which is problematic due to the large number of strains with multidrug resistance among the genus Salmonella.

The third phage has genomic DNA comprising or consisting of a nucleotide sequence of any one of the followings (a) to (c):
(a) the nucleotide sequence of SEQ ID NO: 13;
(b) a nucleotide sequence in which one or a plurality of nucleotides are added, deleted, and/or substituted in the nucleotide sequence of SEQ ID NO: 13; and
(c) a nucleotide sequence having 90% or more sequence identity to the nucleotide sequence of SEQ ID NO: 13.

In particular, the sequence identity defined in (c) is 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

The third phage is useful for treating or preventing food poisoning because it can exhibit a wide range of lytic activity against Salmonella species, particularly S. typhimurium, which is problematic due to the large number of strains with multidrug resistance.

### <Fourth Phage/Lytic Agent>

### (Overview)

One or more embodiments of the present invention provides a phage having lytic activity against a Salmonella species and having a feature below (sometimes referred to as “fourth phage” in the present specification), and a lytic agent comprising the phage (sometimes referred to as “fourth lytic agent” in the present specification). The fourth lytic agent is a lytic agent that exhibits specific lytic activity against particularly a specific Salmonella species, for example, a lytic agent for S. Montevideo. The fourth lytic agent comprises a bacteriophage having a genomic DNA sequence comprising a specific nucleotide sequence.

According to the fourth lytic agent, target bacteria can be lysed and controlled.

### (Feature)

The fourth lytic agent is particularly a lytic agent for a Salmonella species, especially a lytic agent for S. Montevideo. The fourth lytic agent comprises a phage having lytic activity against a Salmonella species and having a feature below.

The fourth phage has a genomic DNA sequence comprising a specific nucleotide sequence.

The present inventors have discovered a phage having lytic activity specifically against a bacterial strain of S. Montevideo and identified the genomic DNA sequence (SEQ ID NO: 14) of the phage.

The fourth phage has a genomic DNA sequence comprising or consisting of a nucleotide sequence of any one of the following (a) to (c):
(a) the nucleotide sequence of SEQ ID NO: 14;
(b) a nucleotide sequence in which one or a plurality of nucleotides are added, deleted, and/or substituted in the nucleotide sequence of SEQ ID NO: 14; and
(c) a nucleotide sequence having 95% or more sequence identity to the nucleotide sequence of SEQ ID NO: 14.

In particular, the sequence identity defined in (c) is 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

Since the fourth phage can exhibit lytic activity specifically against a Salmonella species, particularly S. Montevideo, it is useful, for example, for identifying the serotype of bacteria causing food poisoning.

### <Fifth Phage/Lytic Agent>

### (Overview)

One or more embodiments of the present invention provides a phage having lytic activity against a Salmonella species and having a feature below (sometimes referred to as “fifth phage” in the present specification), and a lytic agent comprising the phage (sometimes referred to as “fifth lytic agent” in the present specification). The fifth lytic agent is a lytic agent that exhibits specific lytic activity particularly against a specific Salmonella species, for example, a lytic agent for S. typhimurium. The fifth lytic agent comprises a bacteriophage having genomic DNA comprising a gene encoding an endonuclease consisting of a specific amino acid sequence.

According to the fifth lytic agent, the target bacteria can be lysed and controlled.

### (Feature)

The fifth lytic agent is in particular a lytic agent for a Salmonella species, in particular, a lytic agent for S. typhimurium. The fifth lytic agent comprises a phage having lytic activity against the Salmonella species and having a feature below.

The fifth phage has genomic DNA comprising a gene encoding an endonuclease that consists of a specific amino acid sequence and has endonuclease activity.

The present inventors have discovered a phage having lytic activity specific to S. typhimurium and identified the genomic DNA sequence (SEQ ID NO: 17) of this phage. The present inventors have further identified a novel endonuclease gene from the genomic DNA sequence of the phage. The amino acid sequence of the endonuclease and the nucleotide sequence encoding it are shown in SEQ ID NOs: 15 and 16, respectively. The endonuclease is involved in shutting down replication of the host genome, which can enhance lytic activity.

### (1) Endonuclease

The endonuclease in the fifth phage consists of an amino acid sequence of any one of the following (a) to (c):
(a) the amino acid sequence of SEQ ID NO: 15;
(b) an amino acid sequence in which one or a plurality of amino acids are added, deleted, and/or substituted in the amino acid sequence of SEQ ID NO: 15; and
(c) an amino acid sequence having 90% or more sequence identity to the amino acid sequence of SEQ ID NO: 15.

In particular, the sequence identity defined in (c) is 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

### (2) Endonuclease Gene

The gene encoding the endonuclease comprises, for example, a nucleotide sequence of any one of the following (d) to (f):
(d) the nucleotide sequence of SEQ ID NO: 16;
(e) a nucleotide sequence in which one or a plurality of nucleotides are added, deleted, and/or substituted in the nucleotide sequence of SEQ ID NO: 16; and
(f) a nucleotide sequence having 90% or more sequence identity to the nucleotide sequence of SEQ ID NO: 16.

Alternatively, a nucleotide sequence that hybridizes under highly stringent conditions to a nucleotide sequence complementary to the nucleotide sequence of SEQ ID NO: 16 can also be mentioned.

In particular, the sequence identity defined in (f) is 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

### (3) Genomic DNA

The fifth phage has genomic DNA comprising a gene encoding an endonuclease.

The genomic DNA sequence comprises or consists of, for example, a nucleotide sequence of any one of the following (g) to (k):
(g) the nucleotide sequence of SEQ ID NO: 17;
(h) a nucleotide sequence in which one or a plurality of nucleotides are added, deleted, and/or substituted in the nucleotide sequence other than the nucleotide sequence of the gene in the nucleotide sequence of SEQ ID NO: 17;
(i) a nucleotide sequence having 80% or more sequence identity to the nucleotide sequence of SEQ ID NO: 17, in the nucleotide sequence other than the nucleotide sequence of the gene;
(j) a nucleotide sequence in which one or a plurality of nucleotides are added, deleted, and/or substituted in the nucleotide sequence of SEQ ID NO: 17; and
(k) a nucleotide sequence having 90% or more sequence identity to the nucleotide sequence of SEQ ID NO: 17.

In particular, the sequence identity defined in (i) is 81% or more, 82% or more, 83% or more, 84% or more, 85% or more, 86% or more, 87% or more, 88% or more, 89% or more, 90% or more, 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

The nucleotide sequence defined in (i) is, in other words, a nucleotide sequence in which the nucleotide sequence other than a gene corresponding to the above-described gene has a sequence identity of 80% or more to the nucleotide sequence other than the nucleotide sequence of the above-described gene in the nucleotide sequence of SEQ ID NO: 17.

In particular, the sequence identity defined in (k) is 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

In one or more embodiments, the fifth phage is characterized by having a genomic DNA sequence comprising a specific nucleotide sequence, and exhibits lytic activity against target bacteria. The genomic DNA sequence of the fifth phage may be a genomic DNA sequence comprising the nucleotide sequence of SEQ ID NO: 17 (47638 bp), a nucleotide sequence in which one or a plurality of nucleotides are added, deleted, and/or substituted in the nucleotide sequence of SEQ ID NO: 17, or a nucleotide sequence having 90% or more, 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more sequence identity to the nucleotide sequence of SEQ ID NO: 17.

The fifth phage can exhibit lytic activity specifically against a Salmonella species, particularly S. typhimurium, and thus is useful, for example, for identifying the serotype of bacteria causing food poisoning.

### <Sixth Phage/Lytic Agent>

### (Overview)

One or more embodiments of the present invention provides a phage having lytic activity against Salmonella species and having a feature below (sometimes referred to as “sixth phage” in the present specification), and a lytic agent comprising the phage (sometimes referred to as “sixth lytic agent” in the present specification). The sixth phage can exhibit lytic activity against S. enteritidis, S. typhimurium, and S. javiana. The sixth phage has a genomic DNA sequence comprising a gene encoding a tail fiber protein consisting of a specific amino acid sequence.

According to the sixth phage, Salmonella species as target bacteria can be lysed and controlled.

### (Feature)

The sixth phage is a phage having lytic activity against Salmonella species and having the following feature.

The sixth phage has genomic DNA comprising a gene encoding a tail fiber protein consisting of a specific amino acid sequence and having recognition activity for target bacteria.

The present inventors have discovered one type of phage having lytic activity against Salmonella species, and identified a tail fiber protein (SEQ ID NO: 18) and a tail fiber gene (SEQ ID NO: 19) from the genomic DNA sequence (SEQ ID NO: 20) of this phage.

The tail fiber protein consists of the amino acid sequence of SEQ ID NO: 18, which consists of 684 amino acid residues. In one or more embodiments of the present invention, the tail fiber protein consisting of the amino acid sequence of SEQ ID NO: 18 can realize highly useful host specificity that is specific to Salmonella species and exhibits lytic activity particularly against S. enteritidis, S. typhimurium, and S. javiana among the genus Salmonella.

### (1) Tail Fiber Protein

The tail fiber protein in the sixth phage consists of an amino acid sequence of any one of the following (a) to (c):
(a) the amino acid sequence of SEQ ID NO: 18;
(b) an amino acid sequence in which one or a plurality of amino acids are added, deleted, and/or substituted in the amino acid sequence of SEQ ID NO: 18; and
(c) an amino acid sequence having 99% or more sequence identity to the amino acid sequence of SEQ ID NO: 18.

In particular, the sequence identity defined in (c) is 99.1% or more, 99.2% or more, 99.3% or more, 99.4% or more, 99.5% or more, 99.6% or more, 99.7% or more, 99.8% or more, or 99.9% or more.

The amino acid sequence of the tail fiber protein of the sixth phage has a plurality of unique amino acid residues that differ from the amino acid sequences of known tail fiber proteins. In the amino acid sequence of the tail fiber protein of the sixth phage (SEQ ID NO: 18), the amino acid corresponding to position 211 is Val, the amino acid corresponding to position 321 is Val, the amino acid corresponding to position 485 is Val, the amino acid corresponding to position 533 is Ala, the amino acid corresponding to position 577 is Ser, and the amino acid corresponding to position 583 is Ser. It is surprising that these amino acid residues differ at many positions despite being highly conserved in the tail fiber proteins of other phages, and this is thought to be linked to the characteristic host range of the sixth phage. Therefore, in the amino acid sequence defined in (b) or (c), in particular, the amino acid corresponding to position 211 is Val, the amino acid corresponding to position 321 is Val, the amino acid corresponding to position 485 is Val, the amino acid corresponding to position 533 is Ala, the amino acid corresponding to position 577 is Ser, and/or the amino acid corresponding to position 583 is Ser. Note that such positions are numbered with the initiating methionine assigned as position 1.

### (2) Tail Fiber Gene

The gene encoding the tail fiber protein comprises, for example, a nucleotide sequence of any one of the following (d) to (f):
(d) the nucleotide sequence of SEQ ID NO: 19;
(e) a nucleotide sequence in which one or a plurality of nucleotides are added, deleted, and/or substituted in the nucleotide sequence of SEQ ID NO: 19; and
(f) a nucleotide sequence having 97% or more sequence identity to the nucleotide sequence of SEQ ID NO: 19.

Alternatively, a nucleotide sequence that hybridizes under highly stringent conditions to a nucleotide sequence complementary to the nucleotide sequence of SEQ ID NO: 19 can also be mentioned.

In particular, the sequence identity defined in (f) is 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

### (3) Genomic DNA

The sixth phage has genomic DNA comprising a gene encoding a tail fiber protein.

The genomic DNA sequence comprises, for example, a nucleotide sequence of any one of the following (g) to (k):
(g) the nucleotide sequence of SEQ ID NO: 20;
(h) a nucleotide sequence in which one or a plurality of nucleotides are added, deleted, and/or substituted in the nucleotide sequence other than the nucleotide sequence of the gene in the nucleotide sequence of SEQ ID NO: 20;
(i) a nucleotide sequence having 90% or more sequence identity to the nucleotide sequence of SEQ ID NO: 20, in the nucleotide sequence other than the nucleotide sequence of the gene;
(j) a nucleotide sequence in which one or a plurality of nucleotides are added, deleted, and/or substituted in the nucleotide sequence of SEQ ID NO: 20; and
(k) a nucleotide sequence having 95% or more sequence identity to the nucleotide sequence of SEQ ID NO: 20.

In particular, the sequence identity defined in (i) is 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

The nucleotide sequence defined in (i) is, in other words, a nucleotide sequence in which the nucleotide sequence other than a gene corresponding to the above-described gene has a sequence identity of 90% or more to the nucleotide sequence other than the nucleotide sequence of the above-described gene in the nucleotide sequence of SEQ ID NO: 20.

In particular, the sequence identity defined in (k) is 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more.

In one or more embodiments, the sixth phage is characterized by having a genomic DNA sequence comprising a specific nucleotide sequence, and exhibits lytic activity against target bacteria. The genomic DNA sequence of the sixth phage may be a genomic DNA sequence comprising the nucleotide sequence of SEQ ID NO: 20 (40784 bp), a nucleotide sequence in which one or a plurality of nucleotides are added, deleted, and/or substituted in the nucleotide sequence of SEQ ID NO: 20, or a nucleotide sequence having 90% or more, 90.5% or more, 91.0% or more, 91.5% or more, 92.0% or more, 92.5% or more, 93.0% or more, 93.5% or more, 94.0% or more, 94.5% or more, 95.0% or more, 95.5% or more, 96.0% or more, 96.5% or more, 97.0% or more, 97.5% or more, 98.0% or more, 98.5% or more, 99.0% or more, 99.1% or more, 99.2% or more, 99.3% or more, 99.4% or more, 99.5% or more, 99.6% or more, 99.7% or more, 99.8% or more, or 99.9% or more sequence identity to the nucleotide sequence of SEQ ID NO: 20.

### (4) Advantages

According to one or more embodiments of the present invention, a novel bacteriophage having lytic activity against Salmonella species can be provided. According to one or more embodiments of the present invention, the tail fiber protein consisting of the amino acid sequence of SEQ ID NO: 18 can realize highly useful host specificity that is specific to Salmonella species and exhibits lytic activity particularly against S. enteritidis, S. typhimurium, and S. javiana among the genus Salmonella. Therefore, the sixth phage is also useful for treating or preventing food poisoning.

### <Seventh Phage/Lytic Agent>

### (Overview)

One or more embodiments of the present invention provides a phage having lytic activity against a Salmonella species and having a feature below (sometimes referred to as “seventh phage” in the present specification), and a lytic agent comprising the phage (sometimes referred to as “seventh lytic agent” in the present specification). The seventh lytic agent is a lytic agent that exhibits specific lytic activity particularly against a specific Salmonella species, for example, a lytic agent for S. enteritidis. The seventh lytic agent comprises a bacteriophage having a genomic DNA sequence comprising a specific nucleotide sequence.

According to the seventh lytic agent, the target bacteria can be lysed and controlled.

### (Feature)

The seventh lytic agent is particularly a lytic agent for a Salmonella species, particularly a lytic agent for S. enteritidis. The seventh lytic agent comprises the seventh phage having lytic activity against the Salmonella species and having the following feature.

The seventh phage has genomic DNA comprising a gene encoding a tail spike protein consisting of a specific amino acid sequence and having recognition activity for target bacteria.

The present inventors have discovered a phage having lytic activity specific to S. enteritidis, and identified the genomic DNA sequence (SEQ ID NO: 23) of the phage. The present inventors have further identified, from the genomic DNA sequence of the phage, a gene encoding a tail spike protein which is considered to determine the host range of the phage. The amino acid sequence of the tail spike protein and the nucleotide sequence encoding it are shown in SEQ ID NOs: 21 and 22, respectively.

### (1) Tail Spike Protein

The tail spike protein in one or more embodiments of the present invention consists of the amino acid sequence of SEQ ID NO: 21.

### (2) Tail Spike Gene

The gene encoding the tail spike protein comprises, for example, the nucleotide sequence of SEQ ID NO: 22.

### (3) Genomic DNA

The seventh bacteriophage has genomic DNA comprising the gene encoding the tail spike protein.

The genomic DNA sequence comprises or consists of, for example, a nucleotide sequence of any one of the following (a) to (e):
(a) the nucleotide sequence of SEQ ID NO: 23;
(b) a nucleotide sequence in which one or a plurality of nucleotides are added, deleted, and/or substituted in the nucleotide sequence other than the nucleotide sequence of the gene in the nucleotide sequence of SEQ ID NO: 23;
(c) a nucleotide sequence having 99% or more sequence identity to the nucleotide sequence of SEQ ID NO: 23, in the nucleotide sequence other than the nucleotide sequence of the gene;
(d) a nucleotide sequence in which one or a plurality of nucleotides are added, deleted, and/or substituted in the nucleotide sequence of SEQ ID NO: 23; and
(e) a nucleotide sequence having 99% or more sequence identity to the nucleotide sequence of SEQ ID NO: 23.

In particular, the sequence identity defined in (c) is 99.1% or more, 99.2% or more, 99.3% or more, 99.4% or more, 99.5% or more, 99.6% or more, 99.7% or more, 99.8% or more, or 99.9% or more.

The nucleotide sequence defined in (c) is, in other words, a nucleotide sequence in which the nucleotide sequence other than a gene corresponding to the above-described gene has a sequence identity of 99% or more to the nucleotide sequence other than the nucleotide sequence of the above-described gene in the nucleotide sequence of SEQ ID NO: 23.

In particular, the sequence identity defined in (e) is 99.1% or more, 99.2% or more, 99.3% or more, 99.4% or more, 99.5% or more, 99.6% or more, 99.7% or more, 99.8% or more, or 99.9% or more.

In one or more embodiments, the seventh phage is characterized by having a genomic DNA sequence comprising a specific nucleotide sequence, and exhibits lytic activity against target bacteria. The genomic DNA sequence of the seventh phage may be a genomic DNA sequence comprising the nucleotide sequence of SEQ ID NO: 23 (39162 bp), a nucleotide sequence in which one or a plurality of nucleotides are added, deleted, and/or substituted in the nucleotide sequence of SEQ ID NO: 23, or a nucleotide sequence having a sequence identity of 99.0% or more, 99.1% or more, 99.2% or more, 99.3% or more, 99.4% or more, 99.5% or more, 99.6% or more, 99.7% or more, 99.8% or more, or 99.9% or more to the nucleotide sequence of SEQ ID NO: 23.

The seventh phage is useful for treating or preventing food poisoning because it can exhibit lytic activity particularly against S. enteritidis, which is the most frequently detected serotype in food poisoning in humans. The seventh phage is also useful, for example, for identifying the serotype of bacteria that cause food poisoning because it can exhibit lytic activity specifically against S. enteritidis.

### 2. Composition

### 2-1. Overview

A second aspect of one or more embodiments of the present invention is a composition, particularly a composition for controlling target bacteria. The composition of one or more embodiments of the present invention is characterized by comprising the bacteriophage or lytic agent according to the first aspect. In the composition of one or more embodiments of the present invention, the target bacteria are particularly Salmonella species. For example, in the case of a composition comprising the first phage or the first lytic agent, the target bacteria are in particular S. enteritidis. In the case of a composition comprising the second phage or the second lytic agent, the target bacteria are in particular S. enteritidis, S. typhimurium, S. infantis, S. Montevideo, and S. javiana. In the case of a composition comprising the third phage or the third lytic agent, the target bacteria are in particular S. typhimurium. In the case of a composition comprising the fourth phage or the fourth lytic agent, the target bacteria are in particular S. Montevideo. In the case of a composition comprising the fifth phage or the fifth lytic agent, the target bacteria are in particular S. typhimurium. In the case of a composition comprising the sixth phage or the sixth lytic agent, the target bacteria are in particular S. enteritidis, S. typhimurium, and S. javiana. In the case of a composition comprising the seventh phage or the seventh lytic agent, the target bacteria are in particular S. enteritidis.

According to the composition of one or more embodiments of the present invention, pharmaceutical compositions, additives (for example, food or beverage additives, feed additives, and drinking water additives), food or beverage products, feed, cleaning agents, disinfectants, sterilizing agents, antibacterial agents, etc. that are safe for the human body, have no adverse effects on the environment, and can control target bacteria, can be provided.

### 2-2. Feature

### (1) Essential Active Ingredient

The composition of one or more embodiments of the present invention contains, as an essential active ingredient, at least one of the bacteriophages and the lytic agents described in the first aspect (i.e., the first phage, the second phage, the third phage, the fourth phage, the fifth phage, the sixth phage, the seventh phage, the first lytic agent, the second lytic agent, the third lytic agent, the fourth lytic agent, the fifth lytic agent, the sixth lytic agent, the seventh lytic agent, or a combination thereof). The composition of one or more embodiments of the present invention can lyse and control the target bacteria through this active ingredient.

Since the specific features of the bacteriophages and the lytic agents have been described in detail in the first aspect, the description thereof is omitted here.

The amount of the bacteriophage or lytic agent in the composition of one or more embodiments of the present invention varies depending on various conditions such as the application of the composition, the target for use, the method of use, the dosage form, and the type of bacteria to be lysed, but is particularly an amount sufficient for the bacteriophage to contact and infect the target bacteria in the target for use. The amount of the bacteriophage or lytic agent in one or more embodiments of the composition of the present invention can be an amount effective for the bacteriophage or lytic agent in the composition of one or more embodiments of the present invention to control the target bacteria, within the scope of common technical knowledge in the art. The titer of the phage in the composition of one or more embodiments of the present invention can be, for example, 1×10¹ to 1×10¹⁵ pfu/mL, 1×10³ to 1×10¹³ pfu/mL, 1×10⁵ to 1×10¹¹ pfu/mL, or 1×10⁷ to 1×10⁹ pfu/mL.

In the composition of one or more embodiments of the present invention, the first to seventh phages or lytic agents may be used alone or in combination of two or more. The composition of one or more embodiments of the present invention can comprise, for example, in addition to the first phage or lytic agent, at least one phage or lytic agent selected from the group consisting of the above-described second to seventh phages or lytic agents as active ingredients in combination. The composition of one or more embodiments of the present invention can comprise, for example, in addition to the second phage or lytic agent, at least one phage or lytic agent selected from the group consisting of the above-described first and third to seventh phages or lytic agents as active ingredients in combination. The composition of one or more embodiments of the present invention can comprise, for example, in addition to the third phage or lytic agent, at least one phage or lytic agent selected from the group consisting of the above-described first, second, and fourth to seventh phages or lytic agents as active ingredients in combination. The composition of one or more embodiments of the present invention can comprise, for example, in addition to the fourth phage or lytic agent, at least one phage or lytic agent selected from the group consisting of the above-described first to third and fifth to seventh phages or lytic agents as active ingredients in combination. The composition of one or more embodiments of the present invention can comprise, for example, in addition to the fifth phage or lytic agent, at least one phage or lytic agent selected from the group consisting of the above-described first to fourth, sixth and seventh phages or lytic agents as active ingredients in combination. The composition of one or more embodiments of the present invention can comprise, for example, in addition to the sixth phage or lytic agent, at least one phage or lytic agent selected from the group consisting of the above-described first to fifth and seventh phages or lytic agents as active ingredients in combination. The composition of one or more embodiments of the present invention can comprise, for example, in addition to the seventh phage or lytic agent, at least one phage or lytic agent selected from the group consisting of the above-described first to sixth phages or lytic agents as active ingredients in combination.

For example, when phages whose target bacteria are different from each other or phages whose target bacteria are the same but recognize different cell surface receptors from each other are combined, a synergistic effect or a complementary effect of the lytic activity can be expected.

### (2) Other Active Ingredients

The composition of one or more embodiments of the present invention can comprise, in addition to the bacteriophage or lytic agent described in the first aspect, one or more other active ingredients having the same pharmacological action as, and/or a different pharmacological action from, that of the bacteriophage or lytic agent, as long as they do not affect the lytic activity of the phage.

The types of other active ingredients are not limited. The other active ingredients may be, for example, phages having lytic activity against the same bacteria as, and/or different bacteria from, the target bacteria of the bacteriophage or lytic agent described in the first aspect. Such a phage may be, for example, a phage having lytic activity against Salmonella species.

In addition, known antibiotics and the like can also be mentioned as other active ingredients.

### (3) Inactive Ingredients

The composition of one or more embodiments of the present invention may further comprise inactive ingredients such as carriers (e.g., solid carriers and liquid carriers), excipients, surfactants, emulsifiers, binders, disintegrants, lubricants, solubilizers, suspending agents, coating agents, colorants, flavoring and odor-masking agents, preservatives, stabilizers, isotonic agents, chelating agents, viscosifiers, thickeners, buffers, and pH adjusters, as long as they do not affect the lytic activity of the bacteriophage or lytic agent described in the first aspect.

### 2-3. Application Target

The application target of the composition of one or more embodiments of the present invention (herein, often simply abbreviated as “target”) is not limited, and examples include: livestock facilities such as poultry farms, pig farms, livestock farms, and dairy farms (including, for example, houses, cages, and soil); food or beverage products or feed; food or beverage processing plants or feed manufacturing plants; apparatuses for processing food or beverage products or feed; containers of food or beverage products or feed; and various vertebrate animals including humans, livestock (horses, cattle, sheep, goats, pigs, chickens, etc.), pets (dogs, cats, rabbits, birds, etc.), and experimental animals (mice, rats, monkeys, etc.).

### 2-4. Form

The composition of one or more embodiments of the present invention may be in the form of a pharmaceutical composition, an additive (for example, a food or beverage additive, a feed additive, or a drinking water additive), a food or beverage product, feed, a cleaning agent, a disinfectant, a sterilizing agent, or an antibacterial agent. Each form is described in detail below.

### (1) Pharmaceutical Composition

The composition of one or more embodiments of the present invention can be a pharmaceutical composition.

The pharmaceutical composition of one or more embodiments of the present invention can be used, for example, for controlling target bacteria in a target. The pharmaceutical composition of one or more embodiments of the present invention can also be used, for example, to treat or prevent the infectious disease caused by target bacteria. In the pharmaceutical composition of one or more embodiments of the present invention, the target bacteria are as described in “2-1. Overview”, and particularly Salmonella species.

As used herein, the term “infectious disease caused by Salmonella species” refers to the disease caused by Salmonella species, and is also referred to as Salmonella infection or salmonellosis. Symptoms of the infectious disease caused by Salmonella species (including S. enteritidis, S. Montevideo and S. typhimurium) include fever, abdominal pain, diarrhea, nausea, queasiness, vomiting, bacteremia, etc. The infectious disease caused by Salmonella species can be, for example, food poisoning.

The pharmaceutical composition of one or more embodiments of the present invention may further comprise, in addition to the bacteriophage or lytic agent described in the first aspect, a pharmaceutically acceptable inactive ingredient (i.e., excipient) as described above.

The pharmaceutical composition of one or more embodiments of the present invention may be formulated into any dosage forms such as solid preparations such as tablets, granules, powders, pills, and capsules, liquid preparations such as solutions, suspensions, and syrups, gels, and aerosols. Note that, when the pharmaceutical composition is used as a liquid preparation, it can also be formulated as a dry product intended to be reconstituted with, for example, physiological saline immediately before use. In addition, in the pharmaceutical composition of one or more embodiments of the present invention, the blending amount of the bacteriophage or lytic agent described in the first aspect can be appropriately set, and the blending amount can be changed depending on the dosage form, the severity of the target's disease, and the like.

The target to which the pharmaceutical composition of one or more embodiments of the present invention is administered may be any vertebrate animal, including humans, domestic animals (horses, cattle, sheep, goats, pigs, chickens, etc.), companion animals (dogs, cats, rabbits, birds, etc.), laboratory animals (mice, rats, monkeys, etc.), etc., and is more particularly a human.

Routes of administration of the pharmaceutical composition of one or more embodiments of the present invention include, but are not limited to, oral, intravenous, rectal, vaginal, topical, and the like.

The dose of the pharmaceutical composition of one or more embodiments of the present invention can be appropriately set in consideration of various factors such as the route of administration and the age, body weight, and symptoms of the subject. The pharmaceutical composition of one or more embodiments of the present invention may be administered once, or may be administered a plurality of times at intervals of several hours to several months.

### (2) Food or Beverage Additive

The composition of one or more embodiments of the present invention can be a food or beverage additive.

The food or beverage additive of one or more embodiments of the present invention can be used, for example, for controlling target bacteria in a food or beverage product. The food or beverage additive of one or more embodiments of the present invention can also be used for imparting a specific action (an action of controlling target bacteria or an action of treatment or prevention of the infectious disease caused by target bacteria) to a food or beverage product by being added to the food or beverage product. In the food or beverage additive of one or more embodiments of the present invention, the target bacteria are as described in “2-1. Overview”, and are particularly Salmonella species.

The food or beverage additive of one or more embodiments of the present invention may further comprise, in addition to the bacteriophage or the lytic agent described in the first aspect, the above-mentioned inactive ingredients acceptable in the production of food or beverage products.

The food or beverage additive of one or more embodiments of the present invention may be in the form of a liquid, a gel, or a dry powder. The type of the food or beverage product, as the target to which the food or beverage additive of one or more embodiments of the present invention is added, is as described in “(4) Food or beverage product”.

The food or beverage additive of one or more embodiments of the present invention can be added to, applied to, or sprayed on a food or beverage product by any appropriate method available to those skilled in the art. For example, the food or beverage additive of one or more embodiments of the present invention may be mixed into a raw material of a food or beverage product during the production of the food or beverage product.

### (3) Feed Additive and Drinking Water Additive

The composition of one or more embodiments of the present invention can be a feed additive or a drinking water additive. The feed additive or drinking water additive of one or more embodiments of the present invention can be used, for example, in rearing livestock and the like.

The feed additive or drinking water additive of one or more embodiments of the present invention can be used, for example, for control of target bacteria in feed or drinking water. The feed additive or drinking water additive of one or more embodiments of the present invention can also be used for imparting a specific action (an action of controlling target bacteria or an action of treating or preventing the infectious disease caused by target bacteria) to feed or drinking water by being added to the feed or drinking water. In the feed additive or drinking water additive of one or more embodiments of the present invention, the target bacteria are as described in “2-1. Overview”, and are particularly Salmonella species.

The feed additive of one or more embodiments of the present invention may further comprise, in addition to the bacteriophage or lytic agent described in the first aspect, the above-mentioned inactive ingredients that are acceptable in the production of feed.

The feed additive of one or more embodiments of the present invention may be in the form of a liquid, a gel or a dry powder. The type of feed to which the feed additive of one or more embodiments of the present invention is added is as described in “(5) Feed”.

The feed additive of one or more embodiments of the present invention can be added to, applied to, or sprayed on the feed by any suitable method available to those skilled in the art. For example, the feed additive of one or more embodiments of the present invention may be mixed with a raw material of feed during the production of the feed.

The drinking water additive of one or more embodiments of the present invention may be in the form of a liquid, a gel or a dry powder. The drinking water, as the target to which the drinking water additive of one or more embodiments of the present invention is added, may be, for example, tap water, well water, groundwater, rainwater, or the like, and is not particularly limited. The drinking water may contain other ingredients (for example, antibiotics, etc.).

The drinking water additive of one or more embodiments of the present invention can be added to drinking water by any suitable method available to those skilled in the art. For example, the drinking water additive of one or more embodiments of the present invention may be mixed with drinking water in a suitable container, or may be mixed with drinking water in a water supply apparatus.

### (4) Food or Beverage Product

The composition of one or more embodiments of the present invention can be a food or beverage product.

The food or beverage product of one or more embodiments of the present invention can be used, for example, for controlling target bacteria in the target. The food or beverage product of one or more embodiments of the present invention can also be used, for example, for treatment or prevention of the infectious disease caused by target bacteria. In the food or beverage product of one or more embodiments of the present invention, the target bacteria are as described in “2-1. Overview”, and are particularly Salmonella species. The infectious disease caused by the target bacteria can be, for example, food poisoning.

The food or beverage product of one or more embodiments of the present invention may further comprise, in addition to the bacteriophage or lytic agent described in the first aspect, the above-mentioned inactive ingredients that are acceptable in the production of the food or beverage product.

The food or beverage product of one or more embodiments of the present invention may be in any form, such as fresh foods (vegetables, fruits, meat, seafood, grains, etc.), processed foods, prepared dishes, confectionery, seasonings, beverages, and functional foods. Examples of the functional foods include foods with health claims including foods for specified health uses (including qualified FOSHU [foods for specified health uses]), foods with function claims, and foods with nutrient function claims, foods for special dietary use, dietary supplements, health supplements, supplements (for example, in various dosage forms such as tablets, coated tablets, sugar-coated tablets, capsules, and liquids), and cosmetic foods (for example, diet foods). The food or beverage product may be prepared in any form such as a solid, a liquid, a mixture, a suspension, a paste, a gel, a powder, a granule, or a capsule. The food or beverage product of one or more embodiments of the present invention can be provided so as to comprise the bacteriophage or the lytic agent described in the first aspect by any appropriate method available to those skilled in the art. Specifically, the food or beverage product of one or more embodiments of the present invention may be produced by encapsulating the bacteriophage or the lytic agent in a capsule, wrapping the bacteriophage or the lytic agent in an edible film, an edible coating agent, or the like, or by mixing (adding) an appropriate excipient or the like with the bacteriophage or the lytic agent and then molding the mixture into an appropriate form such as a tablet. The food or beverage product of one or more embodiments of the present invention may also be produced by processing a composition comprising the bacteriophage or lytic agent of one or more embodiments of the present invention and another food material. The food or beverage product of one or more embodiments of the present invention can also be produced, for example, by blending (adding) the bacteriophage or the lytic agent into various foods (beverages, liquid foods, foods for patients, nutritional foods, frozen foods, processed foods, other commercially available foods, and the like).

### (5) Feed

The composition of one or more embodiments of the present invention can be feed.

The feed of one or more embodiments of the present invention can be used, for example, for controlling target bacteria in a target. The feed of one or more embodiments of the present invention can also be used, for example, for treatment or prevention of the infectious disease caused by target bacteria. In the feed of one or more embodiments of the present invention, the target bacteria are as described in “2-1. Overview”, and are particularly Salmonella species. The infectious disease caused by the target bacteria can be, for example, food poisoning.

The feed of one or more embodiments of the present invention may further comprise, in addition to the bacteriophage or lytic agent described in the first aspect, the above-mentioned inactive ingredients which are acceptable in the manufacture of the feed.

Examples of the feed of one or more embodiments of the present invention include, but are not limited to, pasture grass, straw, miscanthus, hay, silage, grains (corn, barley, wheat, rice, etc.), compound feed, food by-products (okara, brewer's spent grain, bread crumbs, etc.), etc. The feed may also be prepared in any form such as a solid, a liquid, a mixture, a suspension, a paste, a gel, a powder, a granule, a capsule, etc.

The feed of one or more embodiments of the present invention can be provided so as to comprise the bacteriophage or lytic agent described in the first aspect by any suitable method available to those skilled in the art. Specifically, the feed of one or more embodiments of the present invention may be produced by encapsulating the bacteriophage or the lytic agent in a capsule, wrapping the bacteriophage or the lytic agent in an edible film, an edible coating agent, or the like, or by mixing (adding) an appropriate excipient or the like with the bacteriophage or the lytic agent and then molding the mixture into an appropriate form such as a tablet. The feed of one or more embodiments of the present invention may also be produced by processing a composition comprising the bacteriophage or lytic agent of one or more embodiments of the present invention and another feed ingredient. The feed of one or more embodiments of the present invention can also be produced by, for example, blending (adding) the bacteriophage or the lytic agent to various feeds.

### (6) Cleaning Agent, Disinfectant, Sterilizing Agent, or Antibacterial Agent

The composition of one or more embodiments of the present invention can be a cleaning agent, a disinfectant, a sterilizing agent, or an antibacterial agent. As used herein, the term “cleaning agent” refers to a composition intended to remove dirt from an application target. As used herein, the term “disinfectant” means a composition intended to reduce pathogenic microorganisms in an application target to a harmless level. As used herein, the term “sterilizing agent” refers to a composition intended to kill bacteria in an application target. As used herein, the term “antibacterial agent” means a composition intended to reduce bacteria in an application target.

The cleaning agent, disinfectant, sterilizing agent, or antibacterial agent of one or more embodiments of the present invention can be used, for example, for controlling the target bacteria in an application target. In the cleaning agent, disinfectant, sterilizing agent, or antibacterial agent of one or more embodiments of the present invention, the target bacteria are as described in “2-1. Overview”, and are particularly Salmonella species.

The cleaning agent, disinfectant, sterilizing agent, or antibacterial agent of one or more embodiments of the present invention may be in the form of a liquid, a gel, or a dry powder.

The application target that is a target to which the cleaning agent, disinfectant, sterilizing agent, or antibacterial agent of one or more embodiments of the present invention is applied is not limited, and examples include: livestock facilities such as poultry farms, pig farms, livestock farms, and dairy farms (including, for example, houses, cages, and soil); food or beverage products or feed; food or beverage processing plants or feed manufacturing plants; apparatuses for processing food or beverage products or feed; containers of food or beverage products or feed; and various vertebrate animals including humans, livestock (horses, cattle, sheep, goats, pigs, chickens, etc.), pets (dogs, cats, rabbits, birds, etc.), and experimental animals (mice, rats, monkeys, etc.).

The cleaning agent, disinfectant, sterilizing agent, or antibacterial agent of one or more embodiments of the present invention can be used, for example, in a form of being added to, applied to, sprayed on, or dispersed on an application target. The cleaning agent, disinfectant, sterilizing agent, or antibacterial agent of one or more embodiments of the present invention can also be used, for example, in a form in which an application target is immersed.

### 3. Target Bacteria Control Method

### 3-1. Overview

A third aspect of one or more embodiments of the present invention is a method for controlling target bacteria. The target bacteria control method of one or more embodiments of the present invention is characterized in that the bacteriophage or lytic agent described in the first aspect or the composition described in the second aspect is used for controlling target bacteria. In the target bacteria control method of one or more embodiments of the present invention, the target bacteria are particularly Salmonella species. For example, when a composition comprising the first phage or the first lytic agent is used, the target bacteria are particularly S. enteritidis. When a composition comprising the second phage or the second lytic agent is used, the target bacteria are particularly S. enteritidis, S. typhimurium, S. infantis, S. Montevideo, and S. javiana. When a composition comprising the third phage or the third lytic agent is used, the target bacteria are particularly S. typhimurium. When a composition comprising the fourth phage or the fourth lytic agent is used, the target bacteria are particularly S. Montevideo. When a composition comprising the fifth phage or the fifth lytic agent is used, the target bacteria are in particular S. typhimurium. When a composition comprising the sixth phage or the sixth lytic agent is used, the target bacteria are in particular S. enteritidis, S. typhimurium, and S. javiana. When a composition comprising the seventh phage or the seventh lytic agent is used, the target bacteria are in particular S. enteritidis.

According to the control method of one or more embodiments of the present invention, target bacteria can be controlled in an application target.

### 3-2. Method

The target bacteria control method of one or more embodiments of the present invention comprises a contact step as an essential step.

The “contact step” is a step of bringing the bacteriophage or the lytic agent described in the first aspect or the composition described in the second aspect into contact with an application target.

In the present aspect, “contact” refers to direct contact of the bacteriophage or the lytic agent described in the first aspect or the composition described in the second aspect with an application target. More specifically, it means that the bacteriophage or the lytic agent described in the first aspect or the phage in the composition described in the second aspect comes into contact with an application target, particularly a site at risk of contamination by the target bacteria. The purpose of this step is to allow the phage, which is an active ingredient, to infect the target bacteria, whereby the target bacteria undergoes lysis. As a result, the effect of controlling target bacteria can be exhibited.

In the target bacteria control method of one or more embodiments of the present invention, the application target is as described in the second aspect.

In the target bacteria control method of one or more embodiments of the present invention, the contact step can be carried out, for example, by adding, applying, spraying or dispersing the bacteriophage or the lytic agent described in the first aspect or the composition described in the second aspect (particularly, a pharmaceutical composition, a food or beverage additive, a feed additive, a drinking water additive, a cleaning agent, a disinfectant, a sterilizing agent or an antibacterial agent) on an application target, or by immersing an application target in the bacteriophage or lytic agent described in the first aspect or the composition described in the second aspect (particularly, a pharmaceutical composition, a food or beverage additive, a feed additive, a drinking water additive, a cleaning agent, a disinfectant, a sterilizing agent or an antibacterial agent).

The contact step can also be performed by administering the composition described in the second aspect (in particular, a pharmaceutical composition, a food or beverage product, or feed) to an application target.

### 4. Method for Treating or Preventing Infectious Disease Caused by Target Bacteria

### 4-1. Overview

A fourth aspect of one or more embodiments of the present invention is a method for treating or preventing an infectious disease caused by target bacteria. The treatment or prevention method of one or more embodiments of the present invention is characterized by using the bacteriophage or lytic agent described in the first aspect, or the composition described in the second aspect, for the treatment or prevention of an infectious disease caused by target bacteria.

### 4-2. Method

The treatment or prevention method of one or more embodiments of the present invention comprises an administration step as an essential step. The “administration step” is a step of administering the bacteriophage or the lytic agent described in the first aspect or the composition described in the second aspect to a target. In the treatment or prevention method of one or more embodiments of the present invention, the target bacteria are particularly Salmonella species. For example, when a composition comprising the first phage or the first lytic agent is used, the target bacteria are in particular S. enteritidis. When a composition comprising the second phage or the second lytic agent is used, the target bacteria are particularly S. enteritidis, S. typhimurium, S. infantis, S. Montevideo, and S. javiana. When a composition comprising the third phage or the third lytic agent is used, the target bacteria are in particular S. typhimurium. When a composition comprising the fourth phage or the fourth lytic agent is used, the target bacteria are in particular S. Montevideo. When a composition comprising the fifth phage or the fifth lytic agent is used, the target bacteria are in particular S. typhimurium. When a composition comprising the sixth phage or the sixth lytic agent is used, the target bacteria are in particular S. enteritidis, S. typhimurium, and S. javiana. When a composition comprising the seventh phage or the seventh lytic agent is used, the target bacteria are in particular S. enteritidis.

In the treatment or prevention method of one or more embodiments of the present invention, the administration target and the administration method (dose, administration route, and administration frequency) are as described in “2-4. (1) Pharmaceutical composition” above.

### 5. Salmonella Species Identification Method

### 5-1. Overview

A fifth aspect of one or more embodiments of the present invention is a method for identifying a Salmonella species. The identification method of one or more embodiments of the present invention is characterized by identifying a Salmonella species utilizing the host specificity of the bacteriophage or the phage constituting the lytic agent described in the first aspect. The identification method of one or more embodiments of the present invention can be a method for identifying S. enteritidis when using the first phage or the first lytic agent. The identification method of one or more embodiments of the present invention can be a method for identifying S. enteritidis, S. typhimurium, S. infantis, S. Montevideo, and S. javiana when using the second phage or the second lytic agent. The identification method of one or more embodiments of the present invention can be a method for identifying S. typhimurium when using the third phage or the third lytic agent. The identification method of one or more embodiments of the present invention can be a method for identifying S. Montevideo when using the fourth phage or the fourth lytic agent. The identification method of one or more embodiments of the present invention can be a method for identifying S. typhimurium when using the fifth phage or the fifth lytic agent. The identification method of one or more embodiments of the present invention may be a method for identifying S. enteritidis, S. typhimurium, and S. javiana when using the sixth phage or the sixth lytic agent. The identification method of one or more embodiments of the present invention can be a method for identifying S. enteritidis when using the seventh phage or the seventh lytic agent.

According to one or more embodiments of the present invention, it is possible to determine whether or not unidentified bacteria are Salmonella species, and to identify them.

### 5-2. Method

The identification method of one or more embodiments of the present invention comprises a culturing step, a mixing step, a mixture culturing step, and a determination step as essential steps, and further comprises an isolation step as an optional step. Hereinafter, each step will be described.

### (1) Isolation Step

The “isolation step” is a step of isolating test bacteria from a specimen suspected of containing Salmonella species. This step is an optional step and may be performed as necessary.

The term “test bacteria” refers to bacteria which are subjected to the identification method of one or more embodiments of the present invention and whose bacterial species has not been identified.

The specimen may be feces, a food or beverage product, or feed, or may be a swab specimen collected from a livestock facility, a food or beverage product processing plant, a feed manufacturing plant, or the like.

When the amount of Salmonella species in the specimen is expected to be large (for example, when the specimen is the stool of a target with salmonellosis), the specimen can be directly streaked on an agar medium for isolation culture. After the isolation culture, the test bacteria can be isolated by picking a single colony. When the amount of Salmonella species in the specimen is expected to be small (for example, when the specimen is food or a swab specimen), the specimen is inoculated in a culture medium and subjected to enrichment culture, and then the culture solution can be streaked on an agar medium for isolation culture. After the isolation culture, the test bacteria can be isolated in the same manner as described above. When the Salmonella species are expected to be damaged or in a dormant state (for example, when processed food is used as a specimen), pre-enrichment culture may be performed before the enrichment culture.

### (2) Culturing Step

The “culturing step” is a step of culturing the isolated test bacteria to obtain a culture. The method for culturing the test bacteria can be performed by a method known in the art.

The “culture” is obtained by culturing test bacteria, and may be either liquid or solid.

In this step, since the test bacteria are in an unidentified state, it is desirable to use a medium capable of widely culturing bacteria as the medium used in this step. A medium capable of culturing at least Salmonella species, which are the bacteria serving as the identification target of one or more embodiments of the present invention, is used. Such a medium can be, for example, a medium containing one or more components selected from protein enzymatic decomposition products such as peptone and tryptone, biologically derived extracts such as potato dextrose and yeast extract, amino acids such as glutamic acid or salts thereof, sugars such as glucose, sucrose and lactose, and inorganic salts such as sodium chloride, magnesium chloride, potassium dihydrogen phosphate and sodium thiosulfate. Specific examples of the medium and its composition include LB medium (Lysogeny Broth medium; a standard medium containing tryptone, yeast extract, and sodium chloride), DHL medium (Desoxycholate Hydrogen sulfide lactose medium; a medium for Enterobacteriaceae containing desoxycholate, etc.), SS medium (Salmonella-Shigella medium; a selective medium for Salmonella species and Shigella species containing beef extract, peptone, etc.), RV medium (Rappaport-Vassiliadis medium; an enrichment medium for Salmonella species containing peptone, etc.), and others.

The isolated test bacteria are inoculated into the medium and cultured under appropriate culture conditions. The culture conditions are, for example, 20 to 40° C., 20 to 30° C., 22 to 28° C., or 24 to 26° C., and in the case of a liquid medium, the culture can be obtained by culturing while being stirred. The culture time is not limited, but for example, culturing may be performed until the optical density at 600 nm reaches about 1.0. By this step, a culture of the test bacteria is obtained. Alternatively, the culturing may be multi-stage culturing of two or more stages. For example, a soft agar-containing liquid medium is added to a culture solution obtained after culturing in a liquid medium, and the mixture is poured onto a solid medium such as an agar medium and solidified, followed by further culturing.

### (3) Mixing Step

The “mixing step” is a step of mixing the culture obtained in the culturing step with the bacteriophage or lytic agent described in the first aspect to obtain a mixture.

The “mixture” is a mixture of the culture and the bacteriophage or lytic agent, which may be either liquid or solid.

The mixing method is not particularly limited as long as the culture and the bacteriophage or lytic agent can be mixed. The bacteriophage or lytic agent described in the first aspect may be in a solid state, but may also be administered in a liquid state suspended in water or a liquid medium.

If both the culture and the bacteriophage or lytic agent are liquid, the volume ratio of the culture to the lytic agent can be 1:9, 2:8, 3:7, 4:6, 5:5, 6:4, 7:3, 8:2, or 9:1. After administration, the culture and the bacteriophage or lytic agent can be thoroughly mixed by stirring or similar methods. On the other hand, when a soft agar-containing liquid medium is layered as described above, the culture is solid. In this case, a mixture may be obtained by adding dropwise the bacteriophage or lytic agent onto a solid culture such as a gel surface to mix them on the solid medium.

### (4) Mixture Culturing Step

The “mixture culturing step” is a step of culturing the mixture under predetermined conditions.

Note that when the mixture is cultured, a soft agar-containing liquid medium can be added to the mixture, and the mixture can be poured onto a solid medium such as an agar medium and solidified, followed by further culturing.

The basic procedure of this step follows the aforementioned culturing step. In this step, although not limited, it is particularly useful to perform culturing based on a so-called plaque assay method in order to easily determine the presence or absence of lysis of the test bacteria by the bacteriophage in the determination step described below. For example, a part of the mixture can be mixed with a soft agar medium having the same composition, and then poured onto an agar medium having the same composition before the soft agar medium solidifies, and spread over the entire medium. Thereafter, culturing can be performed under the same conditions as in the culturing step.

### (5) Determination Step

The “determination step” is a step of determining that the test bacteria are Salmonella species when the test bacteria after the culturing step are lysed.

The determination of the presence or absence of lysis is not limited. For example, when the determination is based on a plaque assay method, the determination can be made based on the presence or absence of plaque formation. When plaques are present in the soft agar medium which has been spread on the agar medium and solidified after the aforementioned mixture culturing step, it indicates that the test bacteria have been lysed by infection with the bacteriophage of one or more embodiments of the present invention. Therefore, the test bacteria in this case can be determined to be Salmonella species. On the other hand, when the test bacteria grow on the entire agar medium and no plaques are present, it can be determined that the test bacteria are not Salmonella species.

In order to perform more accurate determination, a negative control of mixing with a medium not containing bacteriophage or lytic agent in the mixture culturing step and/or a positive control using an identified Salmonella species from the culturing step in place of the test bacteria may be simultaneously prepared, and it may be confirmed that no plaques are formed in the negative control and that plaques are observed in the positive control.

### 5-3. Advantages

According to the Salmonella species identification method of one or more embodiments of the present invention, it is possible to identify, for example, whether or not food poisoning, diarrhea, vomiting, or the like is caused by Salmonella species. Furthermore, according to the Salmonella species identification method of one or more embodiments of the present invention, the presence or absence of contamination with Salmonella species can be detected.

### EXAMPLES

One or more embodiments of the present invention will be described in more detail below using Examples. However, the technical scope of one or more embodiments of the present invention is not limited to these Examples.

### [Acquisition and Culturing of Salmonella Species]

The bacterial strains used in these Examples (Examples 1 to 7) are listed in Tables shown in Example sections, and the strains owned by Rakuno Gakuen University shown in the Tables are strains of Salmonella species isolated from animals in Japan. The strains listed in the Tables and acquired from National Institute of Animal Health, the National Agriculture and Food Research Organization (NARO) are strains of Salmonella species isolated from chickens and poultry farms in Japan.

The strain IDs in the Tables are identification numbers assigned in the present specification. The serotype of each strain in the Tables was identified from the results of agglutination tests using Salmonella diagnostic immune sera (Denka) on the basis of the antigenic formulae in the Kaufmann-White scheme. Such serotypes were also confirmed by a gene analysis method such as pulsed-field gel electrophoresis (PFGE) or polymerase chain reaction (PCR) as necessary.

For the strains ST1 to 6 in the Tables, their drug susceptibility, PFGE type, and other characteristics have been investigated (Yukino Tamamura, “Molecular Epidemiological Study on Bovine-Derived Salmonella enterica subsp. enterica serovar typhimurium”, Doctoral Thesis, Department of Veterinary Medicine, School of Veterinary Medicine, Rakuno Gakuen University (2015)).

For the culturing of various Salmonella species, a liquid medium (LB Broth) was used, which was prepared by dissolving 10 g of tryptone, 5 g of yeast extract, and 10 g of sodium chloride in 1 L of H₂O and autoclaving it. In addition, as an agar medium, an agar medium (referred to as “LB Agar”) obtained by adding 15 g of agar per 1 L to the above-mentioned LB Broth and autoclaving it was used. Furthermore, as a soft agar medium to be layered on the upper layer of the agar medium, a soft agar medium (referred to as “LB Top Agar”) obtained by adding 5 g of agarose per 1 L to the above-mentioned LB Broth and autoclaving it was used. The soft agar medium was stored at approximately 50° C. and utilized as needed.

Each of the above strains in a dry powder state was suspended in 0.1 mL of the LB Broth, and then streaked on the LB Agar at 25° C. to isolate a single colony. The isolated colony was inoculated into the LB Broth and cultured with shaking at 25° C., which was used as a preculture solution. As the main culturing, the preculture solution was inoculated into the LB Broth and cultured at 25° C. for 10 to 30 hours until the optical density (600 nm) reached about 1.0. The culture broth after the culturing was used directly as the bacterial culture.

### [Isolation and Purification of Phage]

Novel phages were isolated from natural wastewater or soil obtained in Japan. The phage isolation method was performed based on the conventional plaque assay method. First, wastewater from a pond, a lake, or a similar source, or wastewater prepared by suspending soil in water, was filtered through a 0.45 m filter to prepare a phage-containing liquid. Next, the bacterial culture and the phage-containing liquid were mixed in equal amounts and allowed to stand at room temperature for about 10 minutes. Then, 0.2 mL of the bacterial/phage mixture was added to 3 mL of the LB Top Agar, quickly mixed using a vortex mixer, and then poured onto the LB Agar. After the LB Top Agar solidified, culturing was performed under static conditions at 25° C. for about 12 hours. Lysis plaques (Plaques) were formed on the bacterial lawn (Lawn) formed by the culturing. Thereafter, the gel of a plaque portion was aspirated using a tip with a cut end, and a phage having lytic activity against Salmonella species was isolated. Then, the phage-containing liquid containing the isolated phage at a high concentration was used instead of the wastewater, and this procedure was repeated to purify the phage.

The isolated phage was suspended in an SM Buffer and collected as a phage-containing liquid after passing through a 0.2 μm filter. This phage-containing liquid was mixed with the bacterial culture under the above conditions, and the phage was isolated again. This procedure was repeated several times to further purify the phage. The composition of the SM Buffer is shown in the Table below.

TABLE 1

| | | |
|---|---|---|
| SM Buffer | Final | Add to 1 L |

| | | | | |
|---|---|---|---|---|
| NaCl | 0.1 | M | 5.8 | g |
| Mg₂SO₄ · 7H₂O | 10 | mM | 1 | g |
| 1M Tris-HCl PH 8.0 | 50 | mM | 50 | mL |

| | | | |
|---|---|---|---|
| Gelatin | 0.1% | 0.1 | g |

### [Amplification and Purification of Phage]

In order to amplify and purify the isolated and purified phage, a plate lysate (PL) method, which is an amplification method using a plaque assay method, was performed. In order to form many plaques on the LB Agar, a bacterial/phage mixture was prepared, mixed with the LB Top Agar, and then spread on the LB Agar for culturing. Thereafter, 3 mL of the SM Buffer was added to the LB Top Agar on which plaques were formed, the mixture was shaken at 25° C. for about 30 minutes, and the supernatant was passed through a 0.2 m filter to collect a collected solution containing the phage.

To 10 mL of the collected solution, 1 g of PEG 6000 (final concentration: 10%) and 0.4 g of NaCl (final concentration: 4%) were added and dissolved, and rotation was performed overnight at 4° C. using a rotator. Subsequently, the solution was centrifuged at 15,000×g at 4° C. for 60 min, and the supernatant was removed. The collected pellets were resuspended in 0.5 mL of the SM Buffer. Next, 0.5 mL of chloroform was added, and the mixture was vigorously stirred and left on ice for 6 hours. After centrifugation at 8,000×g at 4° C. for 10 min, the upper layer was carefully collected to obtain a phage purified solution. The concentration of such a phage purified solution is generally represented by a titer [PFU/mL] based on the number of plaques (Plaque Forming Unit, PFU) by a plaque assay method, and serves as one of indicators of lytic activity. The titer of the prepared phage purified solution was determined by a plaque assay method using an appropriately diluted solution.

### [Host Range Evaluation of Phage]

The host range of the phage was evaluated by the spot test method. 0.1 mL of the bacterial culture alone was added to 3 mL of the LB Top Agar and mixed; the mixture was then poured onto the LB Agar, spread over the entire plate, and solidified. As the bacterial culture, the bacterial culture of Salmonella species prepared in each Example was used. Then, about 5 L of the phage purified solution was added dropwise onto the plate, and cultured under static conditions at 25° C. for about 12 hours. When the spot where the phage was added dropwise on the plate where the bacterial lawn had formed became transparent (Clear) in a circle (about 1 cm in diameter), it was determined that the phage added dropwise had lytic activity against that bacterial strain.

### [Preparation and Sequencing of Phage Genomic DNA]

The genome of the phage was extracted using a TURBO DNA-free™ kit (Thermo Fisher Scientific). Host bacteria-derived genomic DNA, which becomes a contaminant, was removed by treatment according to the manual included with the kit. Afterwards, phage capsid molecules were degraded by Proteinase K treatment using NucleoSpin (registered trademark) Virus (Machery-Nagel) in accordance with the included manual. A phage genomic DNA solution was prepared through genomic DNA purification using a silica spin column. Subsequently, the concentration of the genomic DNA was measured using a Qubit dsDNA HS Assay kit (Thermo Fisher Scientific), and 50 μL of a genomic DNA solution was prepared to achieve a final concentration of 0.2 ng/L. Next, fragmentation of the phage genome and addition of adaptor sequences by PCR were performed by treatment using Nextera XT DNA Library Prep (Illumina) according to the accompanying manual. Next, electrophoresis was performed using a Bioanalyzer (Agilent Technologies) with an Agilent High Sensitivity DNA Kit (Agilent Technologies), and the average bp size of the sample was measured to determine the concentration of DNA fragments. Finally, a measurement sample was prepared by treatment using a Miseq Reagent kit (Illumina) according to the accompanying manual, and measurement was performed using a next-generation sequencer Miseq (Illumina). Using CLC genomics workbench (Qiagen), preprocessing (trimming, etc.) of the obtained data was performed, followed by de novo assembly to obtain a contig sequence corresponding to the phage genome sequence.

### Example 1: Isolation of First Bacteriophage and its Lytic Activity

### (Purpose)

To isolate a novel bacteriophage with lytic activity against a Salmonella species and to verify its lytic activity against the Salmonella species.

### (Methods and Results)

### (1) Acquisition and Culturing of Salmonella Species

The bacterial strains used in Example 1 are listed in the Table below.

TABLE 2

| Strain | | | |
|---|---|---|---|
| ID | Serotype | Strain name | Source |
| SE6 | S. Entertidis | L-2728 | National Institute of Animal |
| | | | Health, NARO |
| SE8 | S. Entertidis | L-2844 | National Institute of Animal |
| | | | Health, NARO |
| SE12 | S. Entertidis | L-3164 | National Institute of Animal |
| | | | Health, NARO |
| SE17 | S. Entertidis | L-3782 | National Institute of Animal |
| | | | Health, NARO |
| SE18 | S. Entertidis | L-5104 | National Institute of Animal |
| | | | Health, NARO |
| ST1 | S. Typhimurium | HRS-TST-129 | School of Veterinary |
| | | | Medicine, Rakuno |
| | | | Gakuen University |
| ST4 | S. Typhimurium | HRS-KST-31 | School of Veterinary |
| | | | Medicine, Rakuno |
| | | | Gakuen University |
| ST6 | S. Typhimurium | HRS-U1 | School of Veterinary |
| | | | Medicine, Rakuno |
| | | | Gakuen University |
| SI1 | S. Infantis | 07:Hr70A | School of Veterinary |
| | | | Medicine, Rakuno |
| | | | Gakuen University |
| SI3 | S. Infantis | 07:Hr1.5 | School of Veterinary |
| | | | Medicine, Rakuno |
| | | | Gakuen University |
| SM | S. Montevideo | S. Montevideo | School of Veterinary |
| | | No. 1 | Medicine, Rakuno |
| | | | Gakuen University |
| SJ | S. Javiana | L-750 | School of Veterinary |
| | | | Medicine, Rakuno |
| | | | Gakuen University |

### (2) Isolation and Purification of First Phage

According to the method described in the above section [Isolation and purification of phage], seven types of novel phages were isolated from natural wastewater or soil and purified (corresponding to the first phage).

### (3) Amplification and Purification of First Phage

According to the method described in the above section [Amplification and purification of phage], the purified solutions of the first phage were prepared, and the titers were measured. The titers were found to be 10⁸ PFU/mL or more.

### (4) Host Range Evaluation of First Phage

The host range of the first phage was evaluated by the spot test method in accordance with the method described in the above section [Host range evaluation of phage].

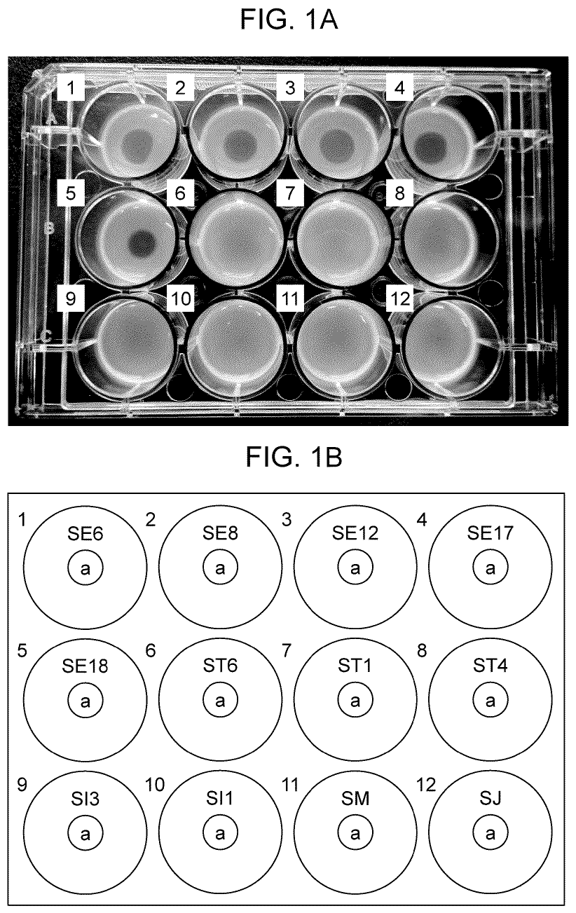

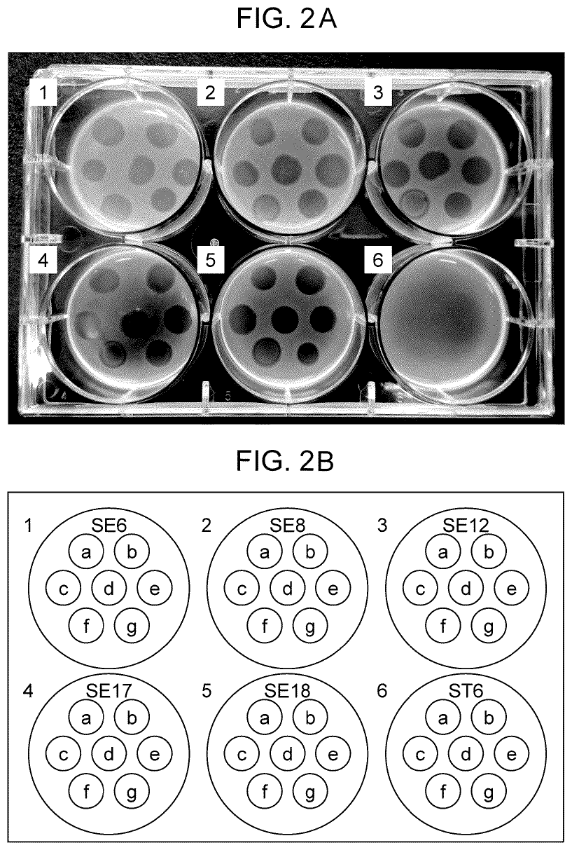

An example of the results is shown in FIGS. 1A, 1B, 2A, and 2B. The seven types of the first phage obtained in this Example exhibited lytic activity against various S. enteritidis bacterial strains, but did not exhibit lytic activity against S. typhimurium, S. infantis, S. Montevideo, and S. javiana.

S. enteritidis is the serotype most frequently detected in food poisoning in humans (Oh and Park, J. Microbiol. Biotechnol. (2017), 27(12), 2075-2088). Thus, the first phage is particularly useful, for example, for treating or preventing food poisoning in humans. In addition, since the first phage exhibited lytic activity specific to S. enteritidis, it is particularly useful for identification of S. enteritidis.

### (5) Genome Analysis of First Phage

The genomic DNA sequences of the first phage were determined and analyzed.

### (i) Preparation and Sequencing of Genomic DNA of First Phage

The genomic DNA sequences of the first phage were determined according to the method described in the above section [Preparation and sequencing of phage genomic DNA]. The determined genomic DNA sequences of the seven types of the first phage are shown in SEQ ID NOs: 1 to 7.

### (ii) Bioinformatics Analysis Based on Genome Sequence Information

The genomic DNA sequences of the first phage (SEQ ID NOs: 1 to 7) had high sequence identity to each other. The sequence identity (Identity) of the shortest genomic DNA sequence of SEQ ID NO: 7 to the genomic DNA sequences of SEQ ID NOs: 1 to 6 was calculated using genetic information processing software GENETYX (https://www.genetyx.co.jp/), and it was 100% over the entire range.

The genomic DNA sequences of SEQ ID NOs: 2 and 7 were used as query sequences to search for similar DNA sequences using the BLAST server provided by NCBI (https: //blast.ncbi.nlm.nih.gov/Blast.cgi). For phages having genomic DNA sequences with high sequence identity over the entire range to SEQ ID NO: 2 or 7, further investigation of prior literature regarding host range was performed. As a result, no phage was found that was known to have a genomic DNA sequence estimated to have a sequence identity of 99% or more to SEQ ID NO: 2 or 7 over the entire range and to have the same host range as the first phage.

### Example 2: Isolation of Second Bacteriophage and its Lytic Activity

### (Purpose)

To isolate a novel bacteriophage having lytic activity against Salmonella species and to verify its lytic activity against the Salmonella species.

### (Methods and Results)

### (1) Acquisition and Culturing of Salmonella Species

The bacterial strains used in Example 2 are listed in the Table below.

TABLE 3

| Strain | | | |
|---|---|---|---|
| ID | Serotype | Strain Name | Source |
| SE1 | S. Entertidis | L-2596 | National Institute of |
| | | | Animal Health, NARO |
| SE2 | S. Entertidis | L-2653 | National Institute of |
| | | | Animal Health, NARO |
| SE3 | S. Entertidis | L-2602 | National Institute of |
| | | | Animal Health, NARO |
| SE4 | S. Entertidis | L-2685 | National Institute of |
| | | | Animal Health, NARO |
| SE5 | S. Entertidis | L-2712 | National Institute of |
| | | | Animal Health, NARO |
| SE6 | S. Entertidis | L-2728 | National Institute of |
| | | | Animal Health, NARO |
| SE7 | S. Entertidis | L-2777 | National Institute of |
| | | | Animal Health, NARO |
| SE8 | S. Entertidis | L-2844 | National Institute of |
| | | | Animal Health, NARO |
| SE9 | S. Entertidis | L-2916 | National Institute of |
| | | | Animal Health, NARO |
| SE10 | S. Entertidis | L-2917 | National Institute of |
| | | | Animal Health, NARO |
| SE11 | S. Entertidis | L-3080 | National Institute of |
| | | | Animal Health, NARO |
| SE12 | S. Entertidis | L-3164 | National Institute of |
| | | | Animal Health, NARO |
| SE13 | S. Entertidis | L-3241 | National Institute of |
| | | | Animal Health, NARO |
| SE14 | S. Entertidis | L-3244 | National Institute of |
| | | | Animal Health, NARO |
| SE15 | S. Entertidis | L-3246 | National Institute of |
| | | | Animal Health, NARO |
| SE16 | S. Entertidis | L-3247 | National Institute of |
| | | | Animal Health, NARO |
| SE17 | S. Entertidis | L-3782 | National Institute of |
| | | | Animal Health, NARO |
| SE18 | S. Entertidis | L-5104 | National Institute of |
| | | | Animal Health, NARO |
| ST3 | S. Typhimurium | HRS-TST-219 | School of Veterinary |
| | | | Medicine, Rakuno |
| | | | Gakuen University |
| SI1 | S. Infantis | 07:Hr70A | School of Veterinary |
| | | | Medicine, Rakuno |
| | | | Gakuen University |
| SI2 | S. Infantis | 07:Hd70B | School of Veterinary |
| | | | Medicine, Rakuno |
| | | | Gakuen University |
| S13 | S. Infantis | 07:Hr1.5 | School of Veterinary |
| | | | Medicine, Rakuno |
| | | | Gakuen University |
| SM | S. Montevideo | S. Montevideo | School of Veterinary |
| | | No.1 | Medicine, Rakuno |
| | | | Gakuen University |
| SJ | S. Javiana | L-750 | School of Veterinary |
| | | | Medicine, Rakuno |
| | | | Gakuen University |

### (2) Isolation and Purification of Second Phage

According to the method described in the above section [Isolation and purification of phage], three types of novel phages were isolated from natural wastewater or soil and purified (corresponding to the second phage).

### (3) Amplification and Purification of Second Phage

According to the method described in the above section [Amplification and purification of phage], purified solutions of the second phage were prepared, and the titers were measured. The titers were found to be 10⁸ PFU/mL or more.

### (4) Host Range Evaluation of Second Phage

The host range of the second phage was evaluated by the spot test method in accordance with the method described in the above section [Host range evaluation of phage].

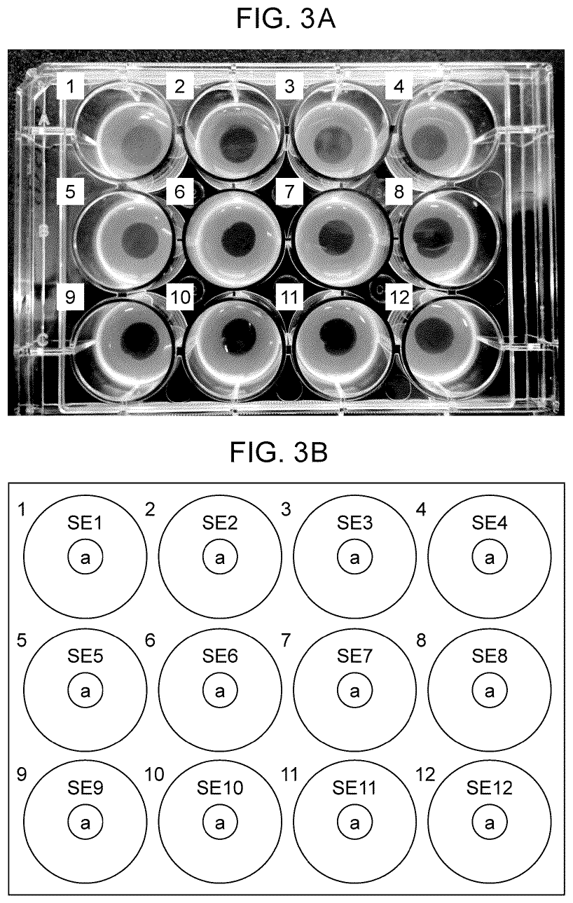

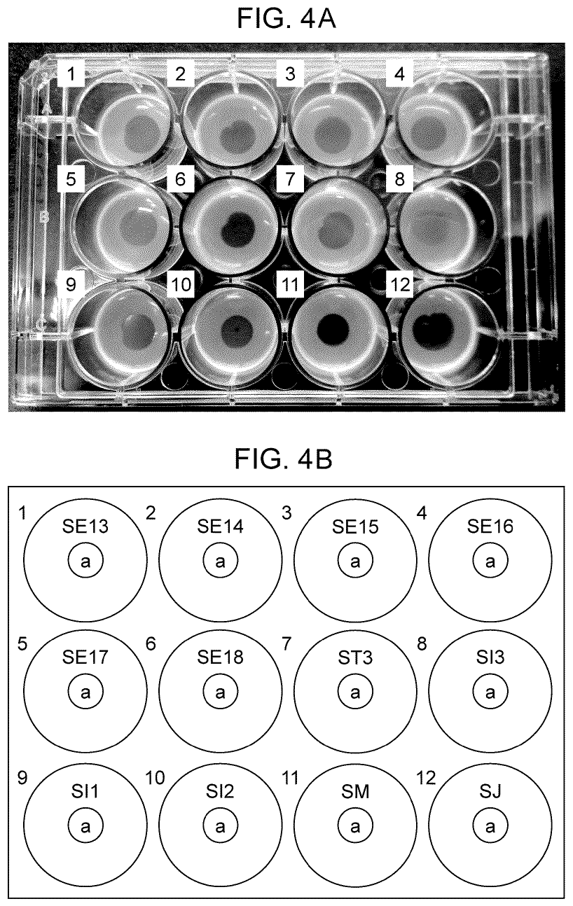

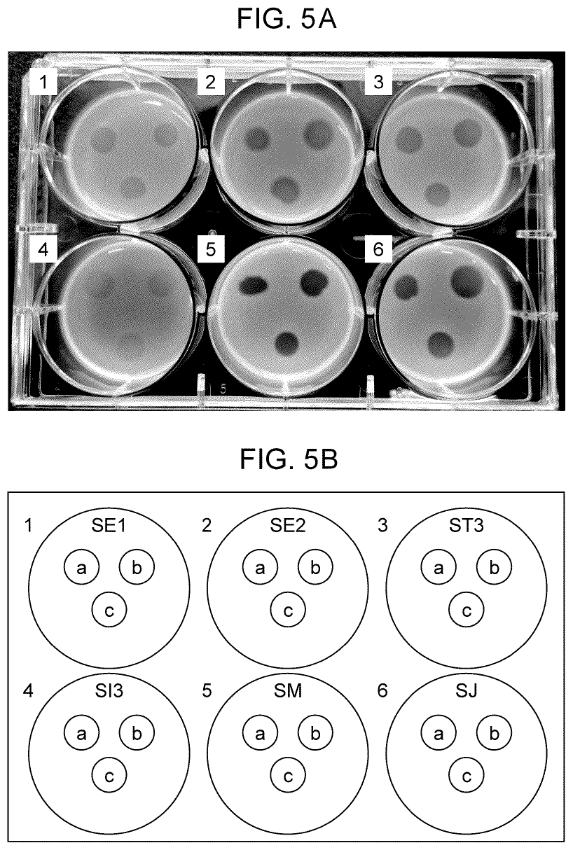

An example of the results is shown in FIGS. 3A, 3B, 4A, 4B, 5A, and 5B. The three types of the second phage obtained in this Example exhibited lytic activity against various bacterial strains, and exhibited lytic activity against all of the tested bacterial strains of S. enteritidis, S. typhimurium, S. infantis, S. Montevideo, and S. javiana. All of these bacterial strains are serotypes frequently detected in food poisoning in humans. Therefore, the second phage is particularly useful, for example, for treating or preventing food poisoning in humans.

### (5) Genome Analysis of Second Phage

The genomic DNA sequences were determined and analyzed for the second phage.

### (i) Preparation and Sequencing of Genomic DNA of Second Phage

The genomic DNA sequences of the second phage were determined according to the method described in the above section [Preparation and sequencing of phage genomic DNA]. The determined genomic DNA sequences of the three types of the second phage are shown in SEQ ID NOs: 10 to 12.

### (ii) Bioinformatics Analysis Based on Genomic Sequence Information

The sequence identity of the genomic DNA sequences (SEQ ID NOs: 10 to 12) of the obtained three types of phages was 99%, and the amino acid sequences of the tail tip proteins were as shown in SEQ ID NO: 8, and they were completely identical to each other.

The genomic DNA sequences (SEQ ID NOs: 10 to 12) of the obtained three types of phages were subjected to search for similar DNA sequences and determination of sequence identity using the BLAST server (https://blast.ncbi.nlm.nih.gov/Blast.cgi) provided by NCBI. As a result of the search, the nucleotide sequence having the highest identity was the genome sequence of Salmonella phage S124 (GenBank accession number: NC_048013.1), and the sequence identity in the entire range was 79.14% (Query Cover/Per.Ident values were 83%/95.36%).

In order to verify the cause of the difference in host range between the obtained three types of phages and S124, the sequences of the tail tip proteins thereof were compared. The tail tip protein genes were identified from the obtained three types of phages. For identification of the genes, the RAST server (https://rast.nmpdr.org/) and the PHASTER server (https://phaster.ca/) were used. As a result, the nucleotide sequence of SEQ ID NO: 9 was identified as the tail tip protein genes.

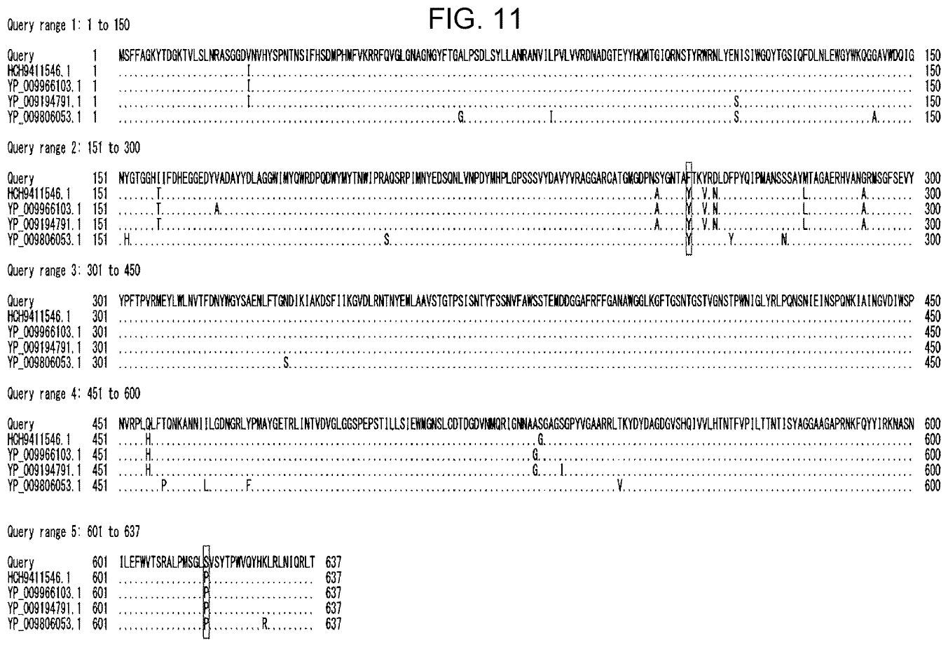

Furthermore, when the amino acid sequence (SEQ ID NO: 8) encoded by the gene comprising the nucleotide sequence of SEQ ID NO: 9 was compared with the homologous protein amino acid sequence of S124 (accession code: YP_009806053.1), the sequence identity was 97.49%. It is highly likely that this difference in the sequences leads to the difference in host range. Note that when using only the amino acid sequence (SEQ ID NO: 8) of the tail tip proteins of the obtained three types of phages as a query sequence to search on the BLAST server provided by NCBI, four known sequences with sequence identities of 95% or more were detected. The alignment between the query sequence and the subject sequences is shown in FIG. 11. In FIG. 11, the amino acid sequence of “HCH9411546.1” is shown in SEQ ID NO: 24, the amino acid sequence of “YP_009966103.1” is shown in SEQ ID NO: 25, the amino acid sequence of “YP_009194791.1” is shown in SEQ ID NO: 26, and the amino acid sequence of “YP_009806053.1” is shown in SEQ ID NO: 27. Among these four known sequences, the sequences other than S124 have no detailed information reported on the host range, or are prophage-derived sequences. It can be seen that F (Phe) at position 258 and S (Ser) at position 617 in the query sequence (the amino acid sequence of the tail tip proteins of the three types of phages obtained) are different from the corresponding residues in the four known sequences described above and are unique amino acid residues found only in the query sequence. These sequence features are presumed to be responsible for the lytic activity of the second phage against a wide range of serotypes.

### Example 3: Isolation of Third Bacteriophage and its Lytic Activity

### (Purpose)

To isolate a novel bacteriophage having lytic activity against a Salmonella species and to verify its lytic activity against the Salmonella species.

### (Methods and Results)

### (1) Acquisition and Culturing of Salmonella Species

The bacterial strains used in Example 3 are listed in the Table below.

TABLE 4

| Strain | | | |
|---|---|---|---|
| ID | Serotype | Strain name | Source |
| ST1 | S. Typhimurium | HRS-TST-129 | School of Veterinary |
| | | | Medicine, Rakuno |
| | | | Gakuen University |
| ST2 | S. Typhimurium | HRS-TST-139 | School of Veterinary |
| | | | Medicine, Rakuno |
| | | | Gakuen University |
| ST3 | S. Typhimurium | HRS-TST-219 | School of Veterinary |
| | | | Medicine, Rakuno |
| | | | Gakuen University |
| ST4 | S. Typhimurium | HRS-KST-31 | School of Veterinary |
| | | | Medicine, Rakuno |
| | | | Gakuen University |

### (2) Isolation and Purification of Third Phage

According to the method described in the above section [Isolation and purification of phage], one type of novel phage was isolated from natural wastewater or soil and purified (corresponding to the third phage).

### (3) Amplification and Purification of Third Phage

According to the method described in the above section [Amplification and purification of phage], a purified solution of the third phage was prepared, and the titer was measured. The titer was found to be 10⁸ PFU/mL or more.

### (4) Host Range Evaluation of Third Phage

The host range of the third phage was evaluated by the spot test method in accordance with the method described in the above section [Host range evaluation of phage].

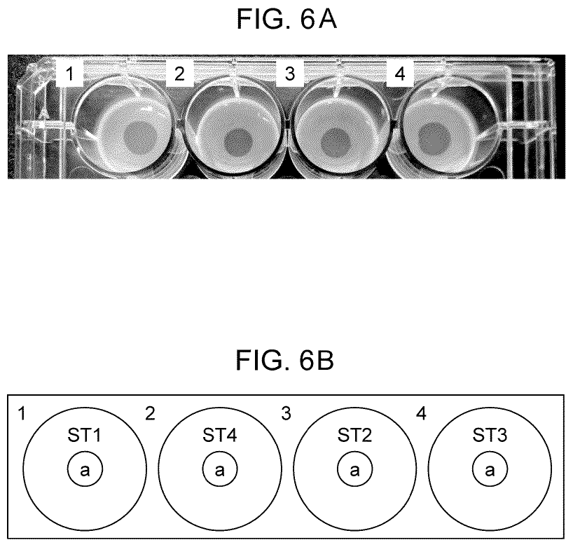

An example of the results is shown in FIGS. 6A and 6B. The one type of the third phage obtained in this Example exhibited lytic activity against various bacterial strains of S. typhimurium. S. typhimurium is a serotype that is frequently detected in food poisoning in humans.

In addition, it is known that S. typhimurium has drug resistance and is difficult to treat with antibiotics. For example, it has been confirmed that the S. typhimurium used in this Example exhibits multidrug resistance to various antibiotics, and specifically, it is known that ST1 is resistant to S/Su, ST4 is resistant to A/C/S/Su/T, ST2 is resistant to A/C/Su, and ST3 is resistant to A/S/Su/T, respectively (Yukino Tamamura, “Molecular Epidemiological Study on Bovine-Derived Salmonella enterica subsp. enterica serovar typhimurium”, Doctoral Thesis, Rakuno Gakuen University, 2015). Note that A represents ampicillin, C represents chloramphenicol, S represents streptomycin, Su represents sulfa drugs, and T represents tetracycline. Therefore, the third phage can effectively control S. typhimurium that is difficult to treat with antibiotics due to drug resistance, and is particularly useful for treatment or prevention of food poisoning in humans.

Furthermore, although not shown in the figure, it was confirmed that the third phage also exhibited lytic activity against ST5 (S. typhimurium HRS-KST-203, School of Veterinary Medicine, Rakuno Gakuen University) and ST6 (S. typhimurium HRS-U1, School of Veterinary Medicine, Rakuno Gakuen University).

### (5) Genome Analysis of Third Phage

The genomic DNA sequence was determined and analyzed for the third phage.

### (i) Preparation and Sequencing of Genomic DNA of Third Phage

The genomic DNA sequence of the third phage was determined according to the method described in the above section [Preparation and sequencing of phage genomic DNA]. The determined genomic DNA sequence of one type of the third phage is shown in SEQ ID NO: 13.

### (ii) Bioinformatics Analysis Based on Genome Sequence Information

Using the BLAST server provided by NCBI (https://blast.ncbi.nlm.nih.gov/Blast.cgi), the genomic DNA sequence of the third phage (SEQ ID NO: 13) was used as a query sequence to search for similar DNA sequences and determine sequence identity. As a result of the search, the most similar nucleotide sequence was the genome sequence of Escherichia phage vB_EcoM-RPN242 (GenBank accession number: OL656110.1), and the sequence identity in the entire range was 87.85% (Query Cover/Per.Ident values were 89%/98.71%). Another one of the most similar nucleotide sequences has a sequence identity of about 85% and is the genome sequence of Escherichia phage vB_EcoM-ZQ1 (GenBank accession number: MW650886.1), and the sequence identity in the entire range was 84.35% (Query Cover/Per.Ident values were 86%/98.09%). Since the hosts of the phages are not Salmonella species, it is suggested that the third phage is a phage having a novel genome sequence for which no homologous genome sequences are known so far.

### Example 4: Isolation of Fourth Bacteriophage and its Lytic Activity

### (Purpose)

To isolate a novel bacteriophage having lytic activity against Salmonella species and to verify its lytic activity against the Salmonella species.

### (Methods and Results)

### (1) Acquisition and Culturing of Salmonella Species

The bacterial strains used in Example 4 are listed in the Table below.

TABLE 5

| Strain | | | |
|---|---|---|---|
| ID | Serotype | Strain name | Source |
| SE1 | S. Entertidis | L-2596 | National Institute of |
| | | | Animal Health, NARO |
| SE2 | S. Entertidis | L-2653 | National Institute of |
| | | | Animal Health, NARO |
| SE8 | S. Entertidis | L-2844 | National Institute of |
| | | | Animal Health, NARO |
| SE10 | S. Entertidis | L-2917 | National Institute of |
| | | | Animal Health, NARO |
| SE11 | S. Entertidis | L-3080 | National Institute of |
| | | | Animal Health, NARO |
| ST2 | S. Typhimurium | HRS-TST-139 | School of Veterinary |
| | | | Medicine, Rakuno |
| | | | Gakuen University |
| ST3 | S. Typhimurium | HRS-TST-219 | School of Veterinary |
| | | | Medicine, Rakuno |
| | | | Gakuen University |
| ST6 | S. Typhimurium | HRS-U1 | School of Veterinary |
| | | | Medicine, Rakuno |
| | | | Gakuen University |
| SI1 | S. Infantis | 07:Hr70A | School of Veterinary |
| | | | Medicine, Rakuno |
| | | | Gakuen University |
| SI3 | S. Infantis | 07:Hr1.5 | School of Veterinary |
| | | | Medicine, Rakuno |
| | | | Gakuen University |
| SJ | S. Javiana | L-750 | School of Veterinary |
| | | | Medicine, Rakuno |
| | | | Gakuen University |
| SM | S. Montevideo | S. Montevideo | School of Veterinary |
| | | No. 1 | Medicine, Rakuno |
| | | | Gakuen University |

### (2) Isolation and Purification of Fourth Phage

According to the method described in the above section [Isolation and purification of phage], a novel phage was isolated from natural wastewater or soil and purified (corresponding to the fourth phage).

### (3) Amplification and Purification of Fourth Phage

According to the method described in the above section [Amplification and purification of phage], a purified solution of the fourth phage was prepared, and the titer was measured. The titer was found to be 10⁸ PFU/mL or more.

### (4) Host Range Evaluation of Fourth Phage

The host range of the fourth phage was evaluated by the spot test method in accordance with the method described in the above section [Host range evaluation of phage].

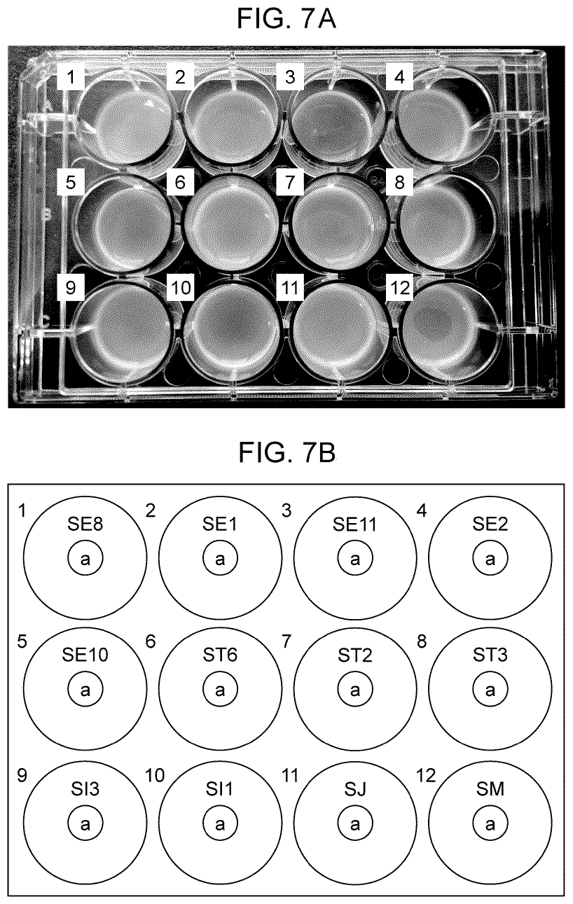

An example of the results is shown in FIGS. 7A and 7B. The fourth phage obtained in this Example exhibited lytic activity against S. Montevideo, but did not exhibit lytic activity against S. enteritidis, S. typhimurium, S. infantis, and S. javiana.

### (5) Genome Analysis of Fourth Phage

The genomic DNA sequence of the fourth phage was determined and analyzed.

### (i) Preparation and Sequencing of Genomic DNA of Fourth Phage

The genomic DNA sequence of the fourth phage was determined according to the method described in the above section [Preparation and sequencing of phage genomic DNA]. The determined genomic DNA sequence of the fourth phage is shown in SEQ ID NO: 14.

### (ii) Bioinformatics Analysis Based on Genome Sequence Information

The genomic DNA sequence of the fourth phage was used as a query sequence to search for similar DNA sequences using the BLAST server provided by NCBI (https://blast.ncbi.nlm.nih.gov/Blast.cgi). As a result of the search, the most similar DNA sequence was the genome sequence of Escherichia coli bacteriophage esc-cop-9 (SEQ ID NO: 1 of U.S. Patent Application Publication No. 2019/0321423), and the sequence identity in the entire range was estimated at 90.68%. However, no phages having a sequence identity of 95% or more over the entire range and using Salmonella species as a host were found.

### Example 5: Isolation of Fifth Bacteriophage and its Lytic Activity

### (Purpose)

To isolate a novel bacteriophage having lytic activity against a Salmonella species and to verify its lytic activity against the Salmonella species.

### (Methods and Results)

### (1) Acquisition and Culturing of Salmonella Species

The bacterial strains used in Example 5 are listed in the Table below.

TABLE 6

| Strain | | | |
|---|---|---|---|
| ID | Serotype | Strain name | Source |
| ST3 | S. Typhimurium | HRS-TST-219 | School of Veterinary |
| | | | Medicine, Rakuno |
| | | | Gakuen University |
| ST4 | S. Typhimurium | HRS-KST-31 | School of Veterinary |
| | | | Medicine, Rakuno |
| | | | Gakuen University |
| ST5 | S. Typhimurium | HRS-KST-203 | School of Veterinary |
| | | | Medicine, Rakuno |
| | | | Gakuen University |
| ST6 | S. Typhimurium | HRS-U1 | School of Veterinary |
| | | | Medicine, Rakuno |
| | | | Gakuen University |

### (2) Isolation and Purification of Fifth Phage

According to the method described in the above section [Isolation and purification of phage], a novel phage was isolated from natural wastewater or soil and purified (corresponding to the fifth phage).

### (3) Amplification and Purification of Fifth Phage

According to the method described in the above section [Amplification and purification of phage], a purified solution of the fifth phage was prepared, and the titer was measured. The titer was found to be 10⁸ PFU/mL or more.

### (4) Host Range Evaluation of Fifth Phage

The host range of the fifth phage was evaluated by the spot test method in accordance with the method described in the above section [Host range evaluation of phage].

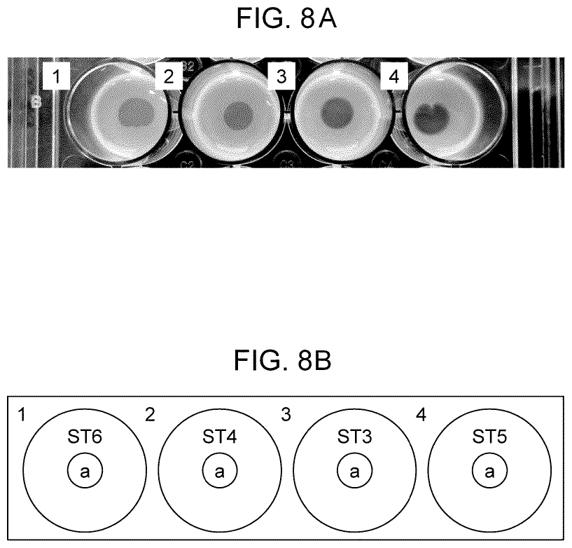

An example of the results is shown in FIGS. 8A and 8B. The fifth phage obtained in this Example exhibited a very high lytic activity against S. typhimurium. Note that the lytic activity against other serotypes of Salmonella species including S. enteritidis was examined by the same method, but the fifth phage did not exhibit lytic activity against the other serotypes of Salmonella species.

### (5) Genome Analysis of Fifth Phage

The genomic DNA sequence of the fifth phage was determined and analyzed.

### (i) Preparation and Sequencing of Genomic DNA of Fifth Phage

The genomic DNA sequence of the fifth phage was determined according to the method described in the above section [Preparation and sequencing of phage genomic DNA]. The determined genomic DNA sequence of the fifth phage is shown in SEQ ID NO: 17.

### (ii) Bioinformatics Analysis Based on Genomic Sequence Information

The genomic DNA sequence of the fifth phage was used as a query sequence to search for similar DNA sequences using the BLAST server provided by NCBI (https://blast.ncbi.nlm.nih.gov/Blast.cgi). As a result of the search, the most similar nucleotide sequence was the genome sequence of Salmonella phage Skate (GenBank accession number: NC 054639.1), and the sequence identity over the entire range was estimated to be 86.61%. As a result of detailed comparison of the genomic DNA sequences of the two phages, it was found that the region corresponding to positions 2385 to 3606 of the genomic DNA sequence of the fifth phage was deleted in Skate. A gene encoding an endonuclease (positions 2434 to 3000 of SEQ ID NO: 17) is present in this region. The amino acid sequence of the endonuclease and the nucleotide sequence encoding it are shown in SEQ ID NOs: 15 and 16, respectively. Nucleases are known to be involved in mechanisms that shut down replication of the host genome. Therefore, it was suggested that the fifth phage having the above-mentioned endonuclease gene can efficiently shut down the replication of the host genome and thus has high lytic activity.

As a result of searching for a similar amino acid sequence on the BLAST server using the amino acid sequence of the endonuclease as a query sequence, there was no genome sequences of phages having a nuclease sequence with a sequence identity of 50% or more. Therefore, the fifth phage was shown to be a novel phage having a novel endonuclease gene.

### Example 6: Isolation of Sixth Bacteriophage and its Lytic Activity

### (Purpose)

To isolate a novel bacteriophage having lytic activity against Salmonella species and to verify its lytic activity against the Salmonella species.

### (Methods and Results)

### (1) Acquisition and Culturing of Salmonella Species

The bacterial strains used in Example 6 are listed in the Table below.

TABLE 7

| Strain | | | |
|---|---|---|---|
| ID | Serotype | Strain name | Source |
| SE1 | S. Entertidis | L-2596 | National Institute of |
| | | | Animal Health, NARO |
| SE4 | S. Entertidis | L-2685 | National Institute of |
| | | | Animal Health, NARO |
| SE6 | S. Entertidis | L-2728 | National Institute of |
| | | | Animal Health, NARO |
| SE8 | S. Entertidis | L-2844 | National Institute of |
| | | | Animal Health, NARO |
| ST1 | S. Typhimurium | HRS-TST-129 | School of Veterinary |
| | | | Medicine, Rakuno |
| | | | Gakuen University |
| ST4 | S. Typhimurium | HRS-KST-31 | School of Veterinary |
| | | | Medicine, Rakuno |
| | | | Gakuen University |
| ST5 | S. Typhimurium | HRS-KST-203 | School of Veterinary |
| | | | Medicine, Rakuno |
| | | | Gakuen University |
| ST6 | S. Typhimurium | HRS-U1 | School of Veterinary |
| | | | Medicine, Rakuno |
| | | | Gakuen University |
| SJ | S. Javiana | L-750 | School of Veterinary |
| | | | Medicine, Rakuno |
| | | | Gakuen University |
| SI1 | S. Infantis | 07:Hr70A | School of Veterinary |
| | | | Medicine, Rakuno |
| | | | Gakuen University |
| S13 | S. Infantis | 07:Hr1.5 | School of Veterinary |
| | | | Medicine, Rakuno |
| | | | Gakuen University |
| SM | S. Montevideo | S. Montevideo | School of Veterinary |
| | | No.1 | Medicine, Rakuno |
| | | | Gakuen University |

### (2) Isolation and Purification of Sixth Phage

According to the method described in the above section [Isolation and purification of phage], one type of novel phage was isolated from natural wastewater or soil and purified (corresponding to the sixth phage).

### (3) Amplification and Purification of Sixth Phage

According to the method described in the above section [Amplification and purification of phage], a purified solution of the sixth phage was prepared, and the titer was measured. The titer was found to be 10⁸ PFU/mL or more.

### (4) Host Range Evaluation of Sixth Phage

The host range of the sixth phage was evaluated by the spot test method in accordance with the method described in the above section [Host range evaluation of phage].

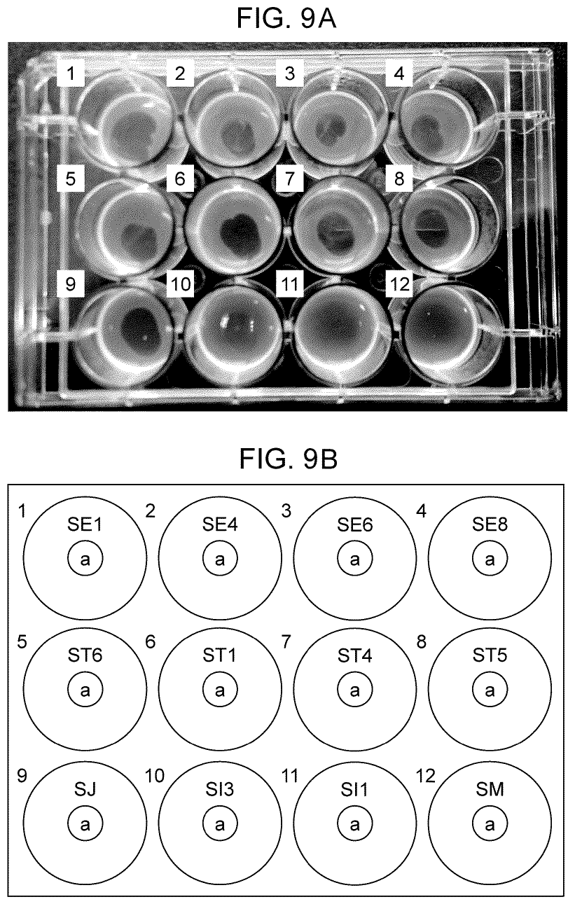

An example of the results is shown in FIGS. 9A and 9B. The one type of the sixth phage obtained in this Example exhibited lytic activity against a plurality of bacterial strains tested, and specifically, exhibited lytic activity against bacterial strains of S. enteritidis, S. typhimurium, and S. javiana. All of these bacterial strains are serotypes that are frequently detected in food poisoning in humans. Therefore, the sixth phage is particularly useful, for example, for the treatment or prevention of food poisoning in humans.

### (5) Genome Analysis of Sixth Phage

The genomic DNA sequence of the sixth phage was determined and analyzed.

### (i) Preparation and Sequencing of Genomic DNA of Sixth Phage

The genomic DNA sequence of the sixth phage was determined according to the method described in the above section [Preparation and sequencing of phage genomic DNA]. The determined genomic DNA sequence of the one type of the sixth phage is shown in SEQ ID NO: 20.

### (ii) Bioinformatics Analysis Based on Genome Sequence Information

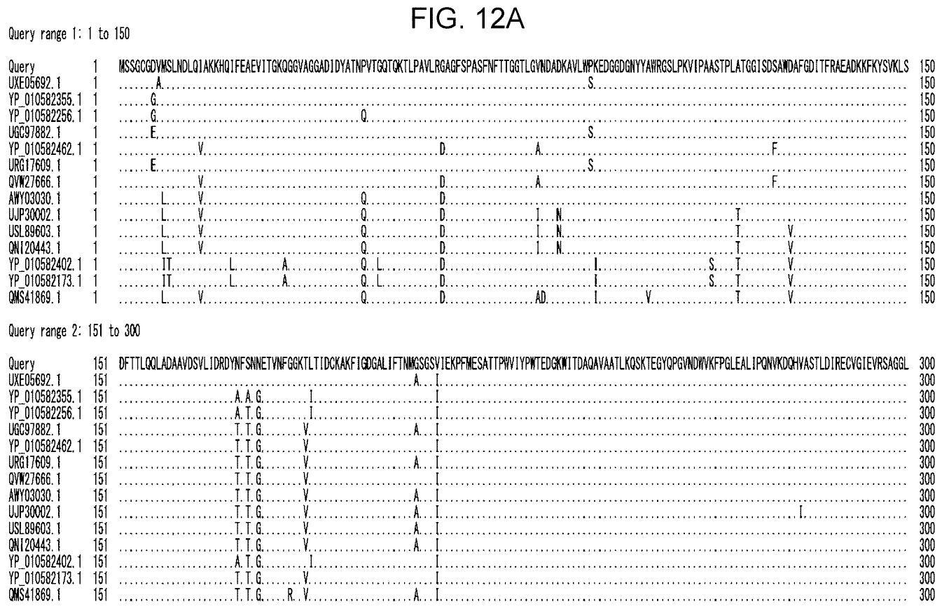

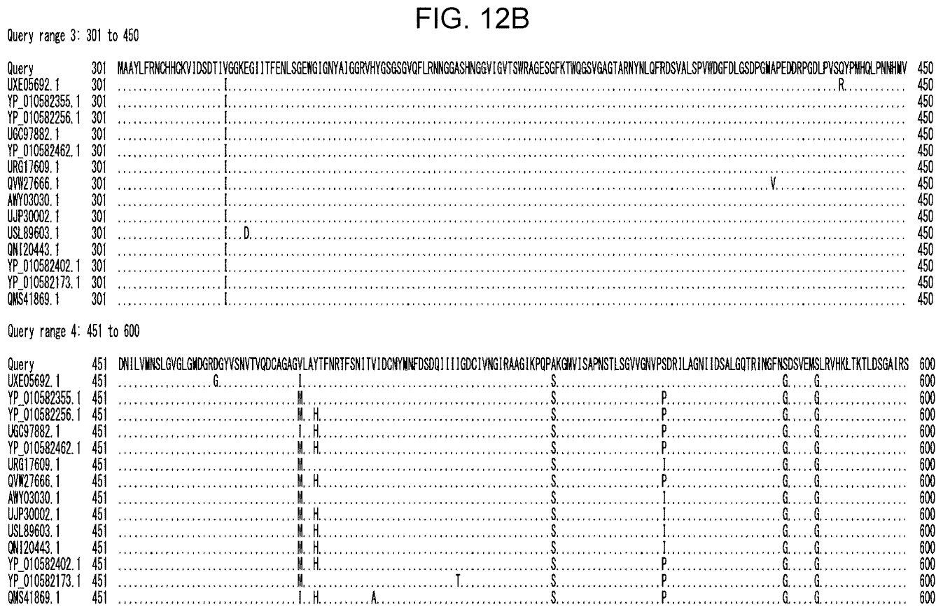

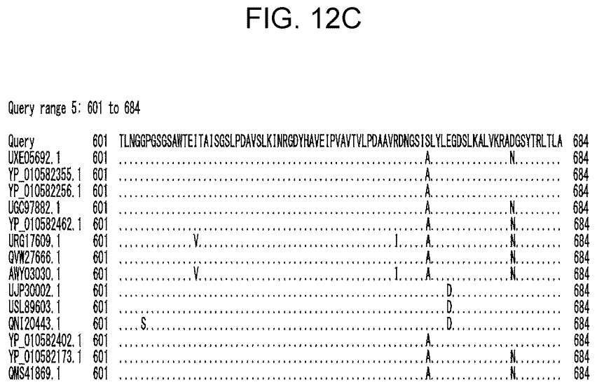

As a result of analysis of the obtained genomic DNA sequence (SEQ ID NO: 20) of the one type of phage, it was found that the amino acid sequence encoded by the nucleotide sequence of positions 32468 to 34522 (CDS) is a tail fiber protein. The amino acid sequence of this tail fiber protein was used as a query sequence to search for similar amino acid sequences and determine sequence identity using the BLAST server (https://blast.ncbi.nlm.nih.gov/Blast.cgi) provided by NCBI. As a result, the amino acid sequences of tail fiber proteins of phages against many Salmonella species were detected, but those with 100% sequence identity were not detected. Accordingly, sequences having an amino acid sequence length of 684 residues, which is the same as that of the tail fiber protein of the sixth phage, and having a sequence identity of 95% or more were extracted by search, and multiple alignment was performed. The results are shown in FIGS. 12A, 12B and 12C. In addition, the phage names, sequence identities, GenBank accession codes, SEQ ID NOs assigned in the present specification, and information on the reactive serotypes (particularly focusing on enteritidis and typhimurium) confirmed from the registration information and literature information of the sequences used for the alignment are shown in the Table below.

TABLE 8

| Salmonella | Identity | GenBank | SEQ | Serotype | | | |
|---|---|---|---|---|---|---|---|
| Phage Name | (%) | Accession No. | ID NO | Enteritidis | Typhimurium | P | U |
| GSP003 | 98.1 | UXE05692.1 | 28 | | | ∘ | |
| vB_SenTO17 | 98.1 | YP_010582355.1 | 29 | x | ∘ | | |
| TS6 | 97.81 | YP_010582256.1 | 30 | | ∘ | | |
| LP31 | 97.51 | UGC97882.1 | 31 | | | ∘ | |
| vB_SenS_SE1 | 97.37 | YP_010582462.1 | 32 | | x | | ∘ |
| GRNsp6 | 97.37 | URG17609.1 | 33 | ∘ | ∘ | | |
| PSDA-2 | 97.22 | QVW27666.1 | 34 | | ∘ | | |
| vB_SpuS_Sp4 | 97.08 | AWY03030.1 | 35 | | | | ∘ |
| CKT1 | 96.78 | UJP30002.1 | 36 | | | ∘ | |
| nctD30 | 96.64 | USL89603.1 | 37 | | | | ∘ |
| SHWT1 | 96.64 | QNI20443.1 | 38 | Δ | ∘ | ∘ | |
| vB_STM-ZS | 96.49 | YP_010582402.1 | 39 | | ∘ | | |
| GRNsp27 | 96.35 | YP_010582173.1 | 40 | ∘ | ∘ | | |
| S55 | 96.05 | QMS41869.1 | 41 | ∘ | | ∘ | |

>75%: ∘, 75-50%: Δ, 50%>: x, P: Pullorum, U: Unknown

As shown in Table 8, even focusing on the two species of enteritidis and typhimurium, it can be said that there is a high possibility that the host ranges of the respective phages are different despite the sequence identity of the tail fiber proteins being 95% or more.

In addition, as can be seen from the results of the amino acid sequence alignment, the tail fiber protein of the sixth phage has a plurality of unique amino acid residues that differ from all the other sequences. They are Val at position 211 (Ile in all the others), Val at position 321 (Ile in all the others), Val at position 485 (Ile or Met in the others), Ala at position 533 (Ser in all the others), Ser at position 577 (Gly in all the others) and Ser at position 583 (Gly in all the others). It is surprising that the different amino acid residues are present at many positions despite being highly conserved in the tail fiber proteins of the other phages, which is thought to be linked to the characteristic host range of the sixth phage.

When the genomic DNA sequence of the sixth phage was also searched using the BLAST server, the phage with the highest sequence identity was Salmonella phage GRNsp27, with a sequence identity of 94.64% (Cover 95%/Ident 99.62%). However, when comparing both sequences in terms of similarity using MUMmer in the genetic analysis software GENETYX (https://www.genetyx.co.jp/), it was found that the Identity of positions 29733 to 34770, corresponding to the region at and around the tail fiber protein gene (positions 32468 to 34522), was as low as 87% (see the Table below). This suggests that the sixth phage is indeed a novel phage significantly different from known phages in the gene region involved in host recognition.

TABLE 9

| | | Identity | Reference | Query | Similarity |
|---|---|---|---|---|---|
| Start | End | (%) | Coverage(%) | Coverage(%) | Errors |
| 1 | 22531 | 99 | 55 | 54 | 113 |
| 22521 | 28331 | 96 | 14 | 14 | 217 |
| 29733 | 34770 | 87 | 12 | 12 | 642 |
| 35384 | 36603 | 97 | 2 | 2 | 31 |
| 37785 | 38379 | 85 | 1 | 1 | 85 |
| 38434 | 38582 | 85 | 0 | 0 | 22 |
| 39683 | 40784 | 98 | 2 | 2 | 13 |

### Example 7: Isolation of Seventh Bacteriophage and its Lytic Activity

### (Purpose)

To isolate a novel bacteriophage having lytic activity against a Salmonella species and to verify its lytic activity against the Salmonella species.

### (Methods and Results)

### (1) Acquisition and Culturing of Salmonella Species

The bacterial strains used in Example 7 are listed in the Table below.

TABLE 10

| Strain | | | |
|---|---|---|---|
| ID | Serotype | Strain name | Source |
| SE11 | S. Entertidis | L-3080 | National Institute of |
| | | | Animal Health, NARO |
| SE13 | S. Entertidis | L-3241 | National Institute of |
| | | | Animal Health, NARO |
| SE14 | S. Entertidis | L-3244 | National Institute of |
| | | | Animal Health, NARO |
| SE15 | S. Entertidis | L-3246 | National Institute of |
| | | | Animal Health, NARO |
| SE16 | S. Entertidis | L-3247 | National Institute of |
| | | | Animal Health, NARO |
| ST3 | S. Typhimurium | HRS-TST-219 | School of Veterinary |
| | | | Medicine, Rakuno |
| | | | Gakuen University |
| ST4 | S. Typhimurium | HRS-KST-31 | School of Veterinary |
| | | | Medicine, Rakuno |
| | | | Gakuen University |
| SI1 | S. Infantis | 07:Hr70A | School of Veterinary |
| | | | Medicine, Rakuno |
| | | | Gakuen University |
| S12 | S. Infantis | 07:Hd70B | School of Veterinary |
| | | | Medicine, Rakuno |
| | | | Gakuen University |
| SI3 | S. Infantis | 07:Hr1.5 | School of Veterinary |
| | | | Medicine, Rakuno |
| | | | Gakuen University |
| SM | S. Montevideo | S. Montevideo No.1 | School of Veterinary |
| | | | Medicine, Rakuno |
| | | | Gakuen University |
| SJ | S. Javiana | L-750 | School of Veterinary |
| | | | Medicine, Rakuno |
| | | | Gakuen University |

### (2) Isolation and Purification of Seventh Phage

According to the method described in the above section [Isolation and purification of phage], a novel phage was isolated from natural wastewater or soil and purified (corresponding to the seventh phage).

### (3) Amplification and Purification of Seventh Phage

According to the method described in the above section [Amplification and purification of phage], a purified solution of the seventh phage was prepared, and the titer was measured. The titer was found to be 10⁸ PFU/mL or more.

### (4) Host Range Evaluation of Seventh Phage

The host range of the seventh phage was evaluated by the spot test method in accordance with the method described in the above section [Host range evaluation of phage].

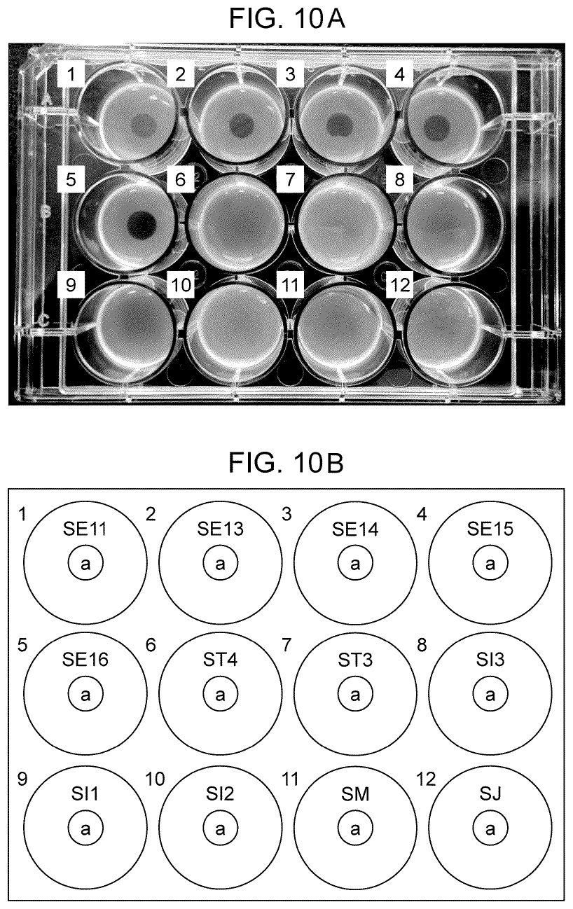

An example of the results is shown in FIGS. 10A and 10B. The seventh phage obtained in this Example exhibited lytic activity against S. enteritidis, but did not exhibit lytic activity against S. typhimurium, S. infantis, S. Montevideo, and S. javiana. S. enteritidis is the serotype most frequently detected in food poisoning in humans (Oh and Park, J. Microbiol. Biotechnol. (2017), 27(12), 2075-2088). Thus, the seventh phage is particularly useful, for example, for treating or preventing food poisoning in humans. In addition, since the seventh phage exhibited lytic activity specifically against S. enteritidis, it is particularly useful for identification of S. enteritidis.

### (5) Genome Analysis of Seventh Phage

The genomic DNA sequence of the seventh phage was determined and analyzed.

### (i) Preparation and Sequencing of Genomic DNA of Seventh Phage

The genomic DNA sequence of the seventh phage was determined according to the method described in the above section [Preparation and sequencing of phage genomic DNA]. The determined genomic DNA sequence of the seventh phage is shown in SEQ ID NO: 23.

### (ii) Bioinformatics Analysis Based on Genomic Sequence Information

The genomic DNA sequence of the seventh phage was used as a query sequence to search for similar DNA sequences using the BLAST server provided by NCBI (https://blast.ncbi.nlm.nih.gov/Blast.cgi). As a result of the search, the most similar nucleotide sequence was the genome sequence of Salmonella phage SPN9CC (GenBank accession number: JF900176.1). Shin et al., Applied and Environmental Microbiology, 2014, vol. 80, No. 1, 374-384 states that SPN9CC exhibited lytic activity against all 7 strains of S. typhimurium. Therefore, SPN9CC clearly has a different host range from the seventh phage which exhibits lytic activity specific to S. enteritidis. When comparing the amino acid sequences of proteins important for host recognition between the seventh phage and SPN9CC, differences were found in the amino acid sequences of the tail spike proteins. Therefore, it was shown that the differences in the amino acid sequences of the tail spike proteins are the cause of the difference in the host range of both phages. Note that the gene encoding the tail spike protein was present at positions 30879 to 32882 of the genomic DNA sequence of the seventh phage. The amino acid sequence of the tail spike protein possessed by the seventh phage is shown in SEQ ID NO: 21, and the nucleotide sequence encoding it is shown in SEQ ID NO: 22.

In the search for similar DNA sequences described above, no phage was found to have a gene encoding a tail spike protein consisting of the amino acid sequence of SEQ ID NO: 21 and to have a genomic DNA sequence with a sequence identity of 99% or more over the entire range with the nucleotide sequence of SEQ ID NO: 23.

All publications, patents and patent applications cited in the present specification are incorporated herein by reference in their entirety.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A method for controlling a Salmonella species, the method comprising bringing a bacteriophage or a lytic agent into contact with an application target.

2. The method of claim 1, wherein the bacteriophage that exhibits lytic activity against the Salmonella species comprises a genomic DNA sequence comprising a nucleotide sequence of any one of (a) to (c) below:
(a) a nucleotide sequence of SEQ ID NO: 13;
(b) a nucleotide sequence in which one or a plurality of nucleotides are added, deleted, and/or substituted in the nucleotide sequence of SEQ ID NO: 13; and
(c) a nucleotide sequence having 90% or more sequence identity to the nucleotide sequence of SEQ ID NO: 13.

3. The method of claim 1, wherein the bacteriophage that exhibits lytic activity against the Salmonella species comprises genomic DNA comprising a gene encoding a tail fiber protein consisting of an amino acid sequence of any one of (a) to (c) below and having recognition activity for target bacteria:
(a) an amino acid sequence of SEQ ID NO: 18;
(b) an amino acid sequence in which one or a plurality of amino acids are added, deleted, and/or substituted in the amino acid sequence of SEQ ID NO: 18; and
(c) an amino acid sequence having 99% or more sequence identity to the amino acid sequence of SEQ ID NO: 18.

4. The method of claim 1, wherein the lytic agent comprises a bacteriophage having a genomic DNA sequence comprising a nucleotide sequence of any one of (a) to (c) below:
(a) the nucleotide sequence of SEQ ID NO: 1;
(b) a nucleotide sequence in which one or a plurality of nucleotides are added, deleted, and/or substituted in the nucleotide sequence of SEQ ID NO: 1; and
(c) a nucleotide sequence having 99% or more sequence identity to the nucleotide sequence of SEQ ID NO: 1.

5. The method of claim 1, wherein the lytic agent comprises a bacteriophage having a genomic DNA sequence comprising a nucleotide sequence of any one of (a) to (c) below:
(a) the nucleotide sequence of SEQ ID NO: 2;
(b) a nucleotide sequence in which one or a plurality of nucleotides are added, deleted, and/or substituted in the nucleotide sequence of SEQ ID NO: 2; and
(c) a nucleotide sequence having 99% or more sequence identity to the nucleotide sequence of SEQ ID NO: 2.

6. The method of claim 1, wherein the lytic agent comprises a bacteriophage having a genomic DNA sequence comprising a nucleotide sequence of any one of (a) to (c) below:
(a) the nucleotide sequence of SEQ ID NO: 3;
(b) a nucleotide sequence in which one or a plurality of nucleotides are added, deleted, and/or substituted in the nucleotide sequence of SEQ ID NO: 3; and
(c) a nucleotide sequence having 99% or more sequence identity to the nucleotide sequence of SEQ ID NO: 3.

7. The method of claim 1, wherein the lytic agent comprises a bacteriophage having a genomic DNA sequence comprising a nucleotide sequence of any one of (a) to (c) below:
(a) the nucleotide sequence of SEQ ID NO: 4;
(b) a nucleotide sequence in which one or a plurality of nucleotides are added, deleted, and/or substituted in the nucleotide sequence of SEQ ID NO: 4; and
(c) a nucleotide sequence having 99% or more sequence identity to the nucleotide sequence of SEQ ID NO: 4.

8. The method of claim 1, wherein the lytic agent comprises a bacteriophage having a genomic DNA sequence comprising a nucleotide sequence of any one of (a) to (c) below:
(a) the nucleotide sequence of SEQ ID NO: 5;
(b) a nucleotide sequence in which one or a plurality of nucleotides are added, deleted, and/or substituted in the nucleotide sequence of SEQ ID NO: 5; and
(c) a nucleotide sequence having 99% or more sequence identity to the nucleotide sequence of SEQ ID NO: 5.

9. The method of claim 1, wherein the lytic agent comprises a bacteriophage having a genomic DNA sequence comprising a nucleotide sequence of any one of (a) to (c) below:
(a) the nucleotide sequence of SEQ ID NO: 6;
(b) a nucleotide sequence in which one or a plurality of nucleotides are added, deleted, and/or substituted in the nucleotide sequence of SEQ ID NO: 6; and
(c) a nucleotide sequence having 99% or more sequence identity to the nucleotide sequence of SEQ ID NO: 6.

10. The method of claim 1, wherein the lytic agent comprises a bacteriophage having a genomic DNA sequence comprising a nucleotide sequence of any one of (a) to (c) below:
(a) the nucleotide sequence of SEQ ID NO: 7;
(b) a nucleotide sequence in which one or a plurality of nucleotides are added, deleted, and/or substituted in the nucleotide sequence of SEQ ID NO: 7; and
(c) a nucleotide sequence having 99% or more sequence identity to the nucleotide sequence of SEQ ID NO: 7.

11. The method of claim 1, wherein the lytic agent comprises a bacteriophage having a genomic DNA sequence comprising a nucleotide sequence of any one of (a) to (c) below:
(a) a nucleotide sequence of SEQ ID NO: 14;
(b) a nucleotide sequence in which one or a plurality of nucleotides are added, deleted, and/or substituted in the nucleotide sequence of SEQ ID NO: 14; and
(c) a nucleotide sequence having 95% or more sequence identity to the nucleotide sequence of SEQ ID NO: 14.

12. The method of claim 1, wherein the lytic agent comprises a bacteriophage having genomic DNA comprising a gene encoding an endonuclease consisting of an amino acid sequence of any one of (a) to (c) below and having endonuclease activity:
(a) an amino acid sequence of SEQ ID NO: 15;
(b) an amino acid sequence in which one or a plurality of amino acids are added, deleted, and/or substituted in the amino acid sequence of SEQ ID NO: 15; and
(c) an amino acid sequence having 90% or more sequence identity to the amino acid sequence of SEQ ID NO: 15.

13. The method of claim 1, wherein the lytic agent comprises a bacteriophage having genomic DNA comprising a gene encoding a tail spike protein consisting of an amino acid sequence of SEQ ID NO: 21, wherein the gene encoding the tail spike protein comprises a nucleotide sequence of any one of (a) to (e) below:
(a) the nucleotide sequence of SEQ ID NO: 23;
(b) a nucleotide sequence in which, in the nucleotide sequence of SEQ ID NO: 23, one or a plurality of nucleotides are added, deleted, and/or substituted in the nucleotide sequence other than the nucleotide sequence of the gene;
(c) a nucleotide sequence having 99% or more sequence identity to the nucleotide sequence of SEQ ID NO: 23, in the nucleotide sequence other than the nucleotide sequence of the gene;
(d) a nucleotide sequence in which one or a plurality of nucleotides are added, deleted, and/or substituted in the nucleotide sequence of SEQ ID NO: 23; and
(e) a nucleotide sequence having 99% or more sequence identity to the nucleotide sequence of SEQ ID NO: 23.

14. The method of claim 2, wherein the Salmonella species is S. typhimurium.

15. The method of claim 4, wherein the Salmonella species is S. enteritidis.

16. The method of claim 11, wherein the Salmonella species is S. Montevideo.

17. The method of claim 12, wherein the Salmonella species is S. typhimurium.

18. The method of claim 13, wherein the Salmonella species is S. enteritidis.

19. The method of claim 3, wherein the Salmonella species is selected from the group consisting of S. enteritidis, S. typhimurium, S. javiana, and combinations thereof.

20. The method of claim 1, wherein the bacteriophage or the lytic agent is contained in a pharmaceutical composition.
